(19) United States
(12) Patent Application Publication
(10) Pub. No.: US 2013/0137852 A1
(43) Pub. Date: May 30, 2013

(54) NOVEL COMPOUNDS AS DPP-IV INHIBITORS AND PROCESS FOR PREPARATION THEREOF

(71) Applicants: Bakulesh Mafatlal Khamar, Ahmedabad (IN); Nirav Kishorbhai Joshi, Ahmedabed (IN); Chandan Hardhan Singh, Ahmedabad (IN); Uday Rajaram Bapat, Ahmedabed (IN); Bipin Dhanjibhai Gadhiya, Ahmedabad (IN); Nirav Sureshbhai Sagar, Ahmedabad (IN); Indravadan Ambalal Modi, Ahmedabad (IN)

(72) Inventors: Bakulesh Mafatlal Khamar, Ahmedabad (IN); Nirav Kishorbhai Joshi, Ahmedabed (IN); Chandan Hardhan Singh, Ahmedabad (IN); Uday Rajaram Bapat, Ahmedabed (IN); Bipin Dhanjibhai Gadhiya, Ahmedabad (IN); Nirav Sureshbhai Sagar, Ahmedabad (IN); Indravadan Ambalal Modi, Ahmedabad (IN)

(73) Assignee: Cadila Corporate Campus, Ahmedabad (IN)

(21) Appl. No.: 13/813,831

(22) PCT Filed: Aug. 3, 2011
(86) PCT No.: PCT/IB11/53443

§ 371 Date: Feb. 1, 2013

(30) Foreign Application Priority Data
Aug. 3, 2010 (IN) 2207/MUM/2010

(51) Int. Cl. C07D 487/04; C07K 1/10; C07D 209/52; C07D 207/16; C07K 5/02

(52) U.S. Cl. CPC C07D 487/04; C07D 207/16; C07K 5/0202; C07D 209/52; C07K 1/10
USPC 530/331; 544/350; 548/540; 548/452; 564/252

(57) ABSTRACT

The present invention relates to process for preparation of novel compounds which are acting as inhibitors of dipeptidyl peptidase-IV enzyme and is depicted by the structural formula as given below: Formula VI. Which are useful in the treatment or prevention of diseases in which the dipeptidylpeptidase-IV enzyme is involved, such as diabetes and particularly type-2 diabetes.

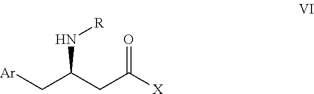

### FIELD OF INVENTION

The present invention relates to process for preparation of novel compounds which are acting as inhibitors of dipeptidyl peptidase-IV enzyme and are useful in the treatment or prevention of diseases in which the dipeptidylpeptidase-IV enzyme is involved, such as diabetes, particularly type-2 diabetes.

### BACKGROUND OF INVENTION

Various amino peptidases are present in the mammals and catalyze the sequential release of peptides from polypeptides such as pyroglutamyl aminopeptidase and prolyaminpeptidase in addition to dipeptidyl peptidase. Dipeptidyl peptidase family includes DPP-II, DPP-IV, DPP-VII, DPP-IX (Curr. Opin. Chem. Biol. 2003, 7, 496). The newly synthesized compounds provide DPP-IV inhibition activity sufficiently fast and with targeted rate. The enzyme DPP-IV also known as CD26 is a cell surface enzyme associated with immune regulation, signal transduction and apoptosis. DPP-IV enzyme works as a suppressor in the development of cancer and tumors. DPP-IV also binds to the enzyme adenosine deaminase specifically and with high affinity.

DPP-IV plays major role in glucose metabolism and is responsible for the degradation of incretins such as Glucagon-like peptide-1 (GLP-1). GLP-1 is an incretin hormone secreted by intestinal L-Cells in response to food intake. The active form of GLP-1 is a 30 amino acid peptides, which stimulate insulin release, inhibits glucagon release and slows gastric emptying; each has benefit in control of glucose homeostasis in patients with type II diabetes. GLP-1 is rapidly degraded by the plasma DPP-IV which cleaves a dipeptide from the N-terminal (Eur. J. Biochem., 1993, 214, 829 and Endocrinology 1995, 136, 3585). DPP-IV inhibitors offer several potential advantages over existing therapies including decreased risk of hypoglycemia, potential weight loss and the potential for regeneration and differentiation of pancreatic β-cells.

WO2010/029422 discloses novel hypoglycemic compounds of formula I and pharmaceutically acceptable salts thereof. The invention relates to novel amino acid derivatives of the formula I,

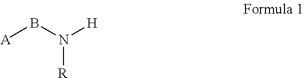

wherein,
A is amino acid, B is peptide bond
R—NH— wherein R is defined in the specification.

Chiral beta amino acid derivatives are reported in the “Journal of bioorganic and medicinal chemistry letters, 17 (2007), 2622-2628” wherein 2-(2,4,5 -trifluorophenyl)acetic acid was coupled with 2,2-dimethyl-1,3-dioxane-4,5-dione (meldrum's acid) using pivaloyl chloride as an activating reagent to produce the coupled product; followed by methanolysis to yield the beta-keto ester. Reaction of beta-keto ester with ammonium acetate produced the corresponding beta-enamino ester which was converted to the chiral beta-(R)-amino esters by asymmetric catalytic hydrogenation with the chloro-(1,5 -cyclooctadiene)rhodium (I) dimmer and (R)-(−)-1-[(S)-2(bis(4-trifluoromethylphenyl)phosphine)ferrocenyl ethyl-di-tert-phosphine under H₂ (100) psi in trifluoroethanol.

Alkyl 4-(2,4,5-trifluorophenyl)-3-(R)-amino butanoate are prepared from commercially available N-Boc protected chiral 3-amino-4-(2,4,5-trifluorophenyl) butanoic acid using C₁ to C₄ alkanol and HCl using known methods.

### OBJECTS OF INVENTION

An object of the invention is to provide novel compounds having DPP-IV inhibitory activity.

Another object of the invention is to provide process for the preparation of novel compounds having DPP-IV inhibitory activity.

Another object of the invention is to provide process for preparation of N-substituted peptide derivatives of DPP-IV inhibitors.

Another object of the invention is to provide novel N-substituted peptide derivatives of 4-(2,4,5-trifluorophenyl)-3-amino butyric acid.

### DESCRIPTION OF DRAWINGS

FIG. 1: In vivo screening results of CPL-2009-0027

FIG. 2: In vivo screening results of CPL-2009-0030

FIG. 3: In vivo screening results of CPL-2009-0031

### DETAIL DESCRIPTION OF THE INVENTION

The present invention provides novel compounds are inhibitors of the dipeptidyl peptidase-IV enzyme and process for preparation thereof.

The general structural formula of compounds having DPP-IV inhibitory activity according to present invention is depicted below:

(Compound of Formula XII)

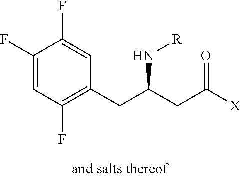

Wherein,

| | | —X is any one from X₁₋₅ | | | | |
|---|---|---|---|---|---|---|
| Nₐ₋ₑ | —R | Nₐ = X₁ | N_{b} = X₂ | N_{c} = X₃ | N_{d} = X₄ | Nₑ = X₅ |
| 1ₐ₋ₑ | 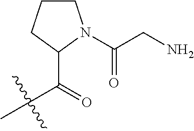 [SMILES: *C(=O)C1CCCN1C(=O)CN] | X₁ | X₂ | X₃ | X₄ | X₅ |
| 2ₐ₋ₑ | 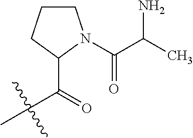 [SMILES: *C(=O)C1CCCN1C(=O)C(C)N] | X₁ | X₂ | X₃ | X₄ | X₅ |
| 3ₐ₋ₑ | 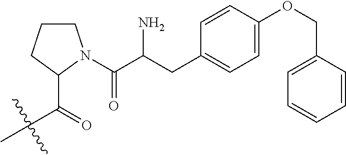 [SMILES: *C(=O)C1CCCN1C(=O)C(N)Cc1ccc(OCc2ccccc2)cc1] | X₁ | X₂ | X₃ | X₄ | X₅ |
| 4ₐ₋ₑ | 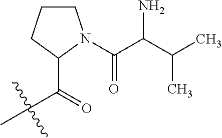 [SMILES: *C(=O)C1CCCN1C(=O)C(N)C(C)C] | X₁ | X₂ | X₃ | X₄ | X₅ |
| 5ₐ₋ₑ | 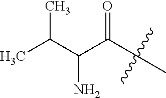 [SMILES: *C(=O)C(N)C(C)C] | X₁ | X₂ | X₃ | X₄ | X₅ |
| 6ₐ₋ₑ | 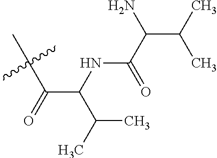 [SMILES: *C(=O)C(NC(=O)C(N)C(C)C)C(C)C] | X₁ | X₂ | X₃ | X₄ | X₅ |
| 7ₐ₋ₑ | 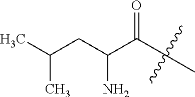 [SMILES: *C(=O)C(N)CC(C)C] | X₁ | X₂ | X₃ | X₄ | X₅ |
| 8ₐ₋ₑ | 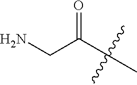 [SMILES: *C(=O)CN] | X₁ | X₂ | X₃ | X₄ | X₅ |
| 9ₐ₋ₑ | 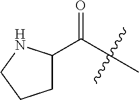 [SMILES: *C(=O)C1CCCN1] | X₁ | X₂ | X₃ | X₄ | X₅ |
| 10ₐ₋ₑ | 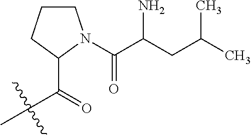 [SMILES: *C(=O)C1CCCN1C(=O)C(N)CC(C)C] | X₁ | X₂ | X₃ | X₄ | X₅ |
| 11ₐ₋ₑ | 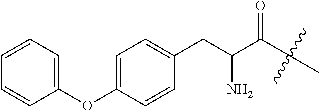 [SMILES: *C(=O)C(N)Cc1ccc(Oc2ccccc2)cc1] | X₁ | X₂ | X₃ | X₄ | X₅ |
| 12ₐ₋ₑ | 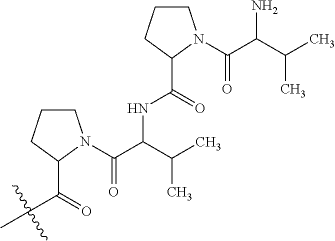 [SMILES: *C(=O)C1CCCN1C(=O)C(NC(=O)C1CCCN1C(=O)C(N)C(C)C)C(C)C] | X₁ | X₂ | X₃ | X₄ | X₅ |
| 13ₐ₋ₑ | 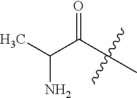 [SMILES: *C(=O)C(C)N] | X₁ | X₂ | X₃ | X₄ | X₅ |
| 14ₐ₋ₑ | 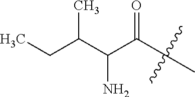 [SMILES: *C(=O)C(N)C(C)CC] | X₁ | X₂ | X₃ | X₄ | X₅ |
| 15ₐ₋ₑ | 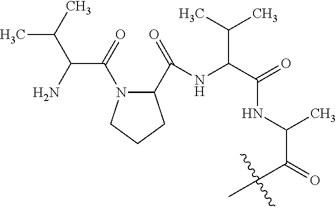 [SMILES: *C(=O)C(C)NC(=O)C(NC(=O)C1CCCN1C(=O)C(N)C(C)C)C(C)C] | X₁ | X₂ | X₃ | X₄ | X₅ |
| 16ₐ₋ₑ | 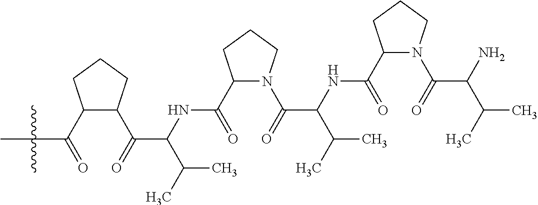 [SMILES: *C(=O)C1CCCC1C(=O)C(NC(=O)C1CCCN1C(=O)C(NC(=O)C1CCCN1C(=O)C(N)C(C)C)C(C)C)C(C)C] | X₁ | X₂ | X₃ | X₄ | X₅ |
| 17ₐ₋ₑ | 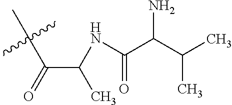 [SMILES: *C(=O)C(C)NC(=O)C(N)C(C)C] | X₁ | X₂ | X₃ | X₄ | X₅ |
| 18ₐ₋ₑ | 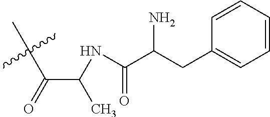 [SMILES: *C(=O)C(C)NC(=O)C(N)Cc1ccccc1] | X₁ | X₂ | X₃ | X₄ | X₅ |
| 19ₐ₋ₑ | 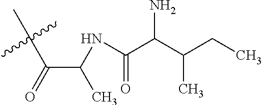 [SMILES: *C(=O)C(C)NC(=O)C(N)C(C)CC] | X₁ | X₂ | X₃ | X₄ | X₅ |
| 20ₐ₋ₑ | 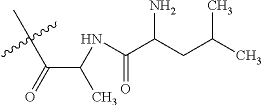 [SMILES: *C(=O)C(C)NC(=O)C(N)CC(C)C] | X₁ | X₂ | X₃ | X₄ | X₅ |
| 21ₐ₋ₑ | 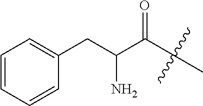 [SMILES: *C(=O)C(N)Cc1ccccc1] | X₁ | X₂ | X₃ | X₄ | X₅ |
| 22ₐ₋ₑ | 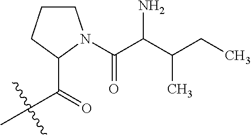 [SMILES: *C(=O)C1CCCN1C(=O)C(N)C(C)CC] | X₁ | X₂ | X₃ | X₄ | X₅ |
| 23ₐ₋ₑ | 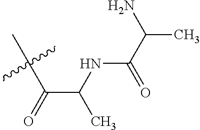 [SMILES: *C(=O)C(C)NC(=O)C(C)N] | X₁ | X₂ | X₃ | X₄ | X₅ |
| 24ₐ₋ₑ | 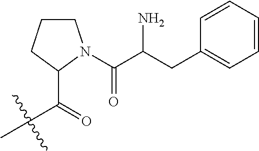 [SMILES: *C(=O)C1CCCN1C(=O)C(N)Cc1ccccc1] | X₁ | X₂ | X₃ | X₄ | X₅ |
| 25ₐ₋ₑ | 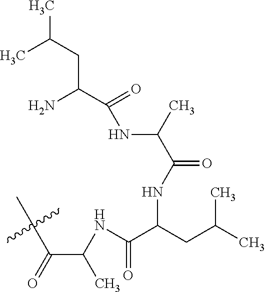 [SMILES: *C(=O)C(C)NC(=O)C(CC(C)C)NC(=O)C(C)NC(=O)C(N)CC(C)C] | X₁ | X₂ | X₃ | X₄ | X₅ |
| 26ₐ₋ₑ | 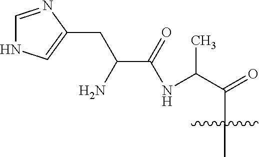 [SMILES: *C(=O)C(C)NC(=O)C(N)Cc1c[nH]cn1] | X₁ | X₂ | X₃ | X₄ | X₅ |

The above novel compounds were prepared by process as depicted in scheme 1:

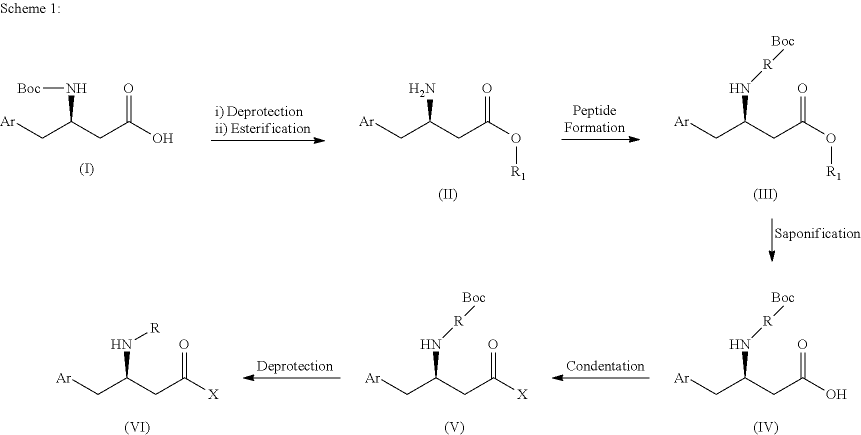

### Wherein,

Ar is phenyl or substituted phenyl having 1 to 5 halogen atoms in the phenyl ring, preferably Ar is 2,4,5-trifluoro phenyl;
R is aa1 or aa1-aa2 or aa1-aa2-aa3 or aa-aa2-aa3-aa4, wherein aa1, aa2, aa3 and aa4 are amino acids selected independently and is linked through a peptide bond;
R₁ is C₁ to C₄ alkyl;
X is defined as any of the substructures X₁ to X₅, wherein substructures X₁ to X₅ are structurally depicted below:

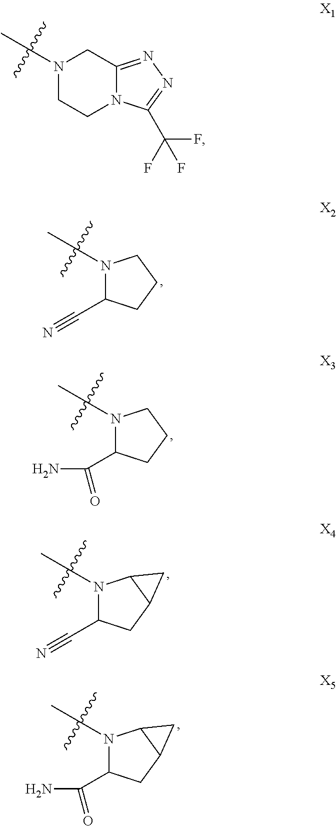 [SMILES: *N1CCn2c(nnc2C(F)(F)F)C1 ; *N1CCCC1C#N ; *N1CCCC1C(N)=O ; *N1C(C#N)CC2CC21 ; *N1C(C(N)=O)CC2CC21]

—NH₂ (X₆),

—NHPh (X₇),

—NHBn (X₈), or

—OH (x₉).

The amino acid (aa1/aa2/aa3/aa4) is selected from any of the following:

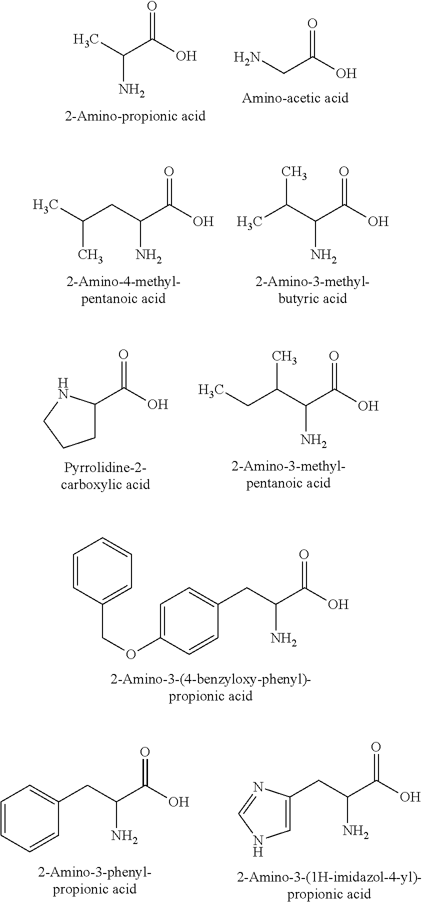

The amino acids as described in the above table provides mono, di, tri or tetra peptide derivatives which on further reaction provides novel DPP-IV inhibitors.

The compounds of formula-VI, wherein X is —NH₂ (X₆), —NHPh (X₇) (Ph is Phenyl), —NHBn (X₈) (Bn is Benzyl) are prepared by using standard conditions by reaction of the compounds of formula—(III) or (IV) with suitable compounds having amino groups.

The compounds are optionally converted to its corresponding pharmaceutically acceptable salt form using acids. The term “pharmaceutically acceptable salts” refers to salts prepared from pharmaceutically acceptable non-toxic acids including inorganic or organic acids. The corresponding salt of compound of formula XII is also prepared from pharmaceutically acceptable non-toxic acids, including inorganic and organic acids by the know method in the art. Using acids such as acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, tartaric acids and the like.

The process for the preparation of compounds according to present invention using sequence of reactions as depicted in scheme 1 comprises reaction of 4-aryl-3-N-peptide substituted beta-amino acid derivatives [compounds of formula—(VI)] wherein,
1) deprotection followed by esterification of N-protected beta-amino acid (formula I) to give chiral beta amino acid ester derivatives (formula II);
2) reaction of chiral beta amino acid ester derivatives (formula II) with carboxyl terminal of N′-protected peptide compounds to give beta-amino N-acyl peptide compounds (formula III);
3) reaction of beta-amino N-protected compound (formula III) with saponifying agent to provide beta-amino N-acylated-4-(2,4,5-trifluorophenyl)-3-amino butyric acid (formula IV);
4) condensation of beta-amino N-acylated-4-(2,4,5-trifluorophenyl)-3-amino butyric acid (formula IV) with any of substructures X₁ to X₅ to give corresponding amides of beta-amino N-acylated-N′-protected peptide compounds (formula V);
5) deprotection of amides of beta-amino N-acylated-N′-protected peptide compounds (formula V) to give 4-aryl-3-N-peptide substituted amino acid derivatives (formula VI).
   The reaction scheme 2 for the preparation of novel compounds, wherein Ar is 2,4,5-trifluoro phenyl is as depicted below:

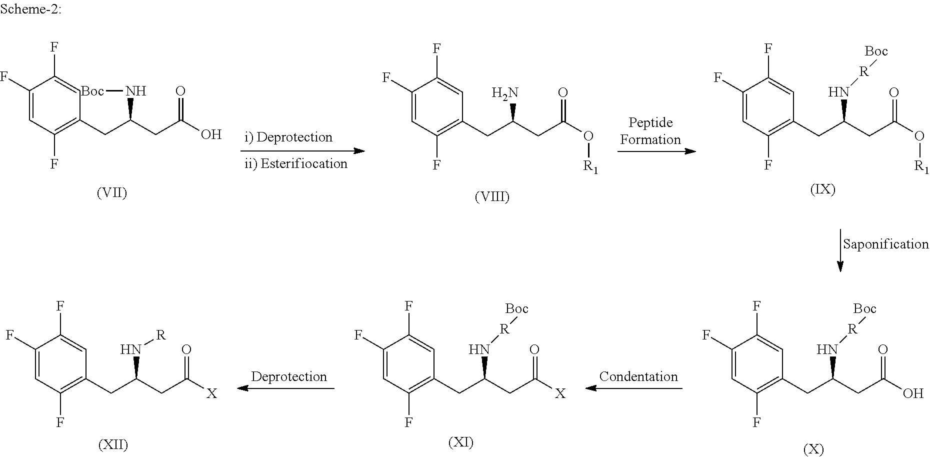

In accordance with the present invention, preparation of novel 4-(2,4,5-trifluorophenyl)-3-(N-peptide Substituted) amino acid derivatives of formula—XII comprises:
1) deprotection followed by esterification of N-protected beta-amino acid (formula VII) to give chiral beta amino acid ester derivatives (formula VIII);
2) reaction of chiral beta amino acid ester derivative (formula VIII) with carboxyl terminal of N′-protected peptide compounds to give beta-amino N-acylated peptide compounds (formula IX), wherein As is 2,4,5-trifluoro phenyl;
3) beta-amino N-acylated peptide compounds (formula IX) are converted into beta-amino N-acylated-4-(2,4,5-trifluorophenyl) butanoic acids (formula X) by saponification;
4) reacting compounds of formula (X) with any of the compound (a) to (e) to give corresponding amides of formula (XI);
5) deprotection of beta-amino N-acylated-N′-protected peptide compounds of formula (XI) to give 4-aryl-3-N-peptide substituted amino acid derivatives (formula XII).

Peptide bond is formed by coupling between a carboxyl group and an amino group in presence of coupling agents. The coupling agents are selected from DCC, EDC, DIC and like. The preferable coupling agents are DCC and EDC.

The saponification reaction is carried out by using Inorganic base. The Inorganic base is selected from NaOH, KOH, LiOH and like. The preferable Inorganic bases are NaOH and LiOH. Deprotection reaction is carried out by using any prior art method.

The present invention is further illustrated with following non-limiting examples:

### EXAMPLE-1

### Preparation of 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-3-phenyl-propionamide Hydrochloric Acid (Compound of Formula 6):

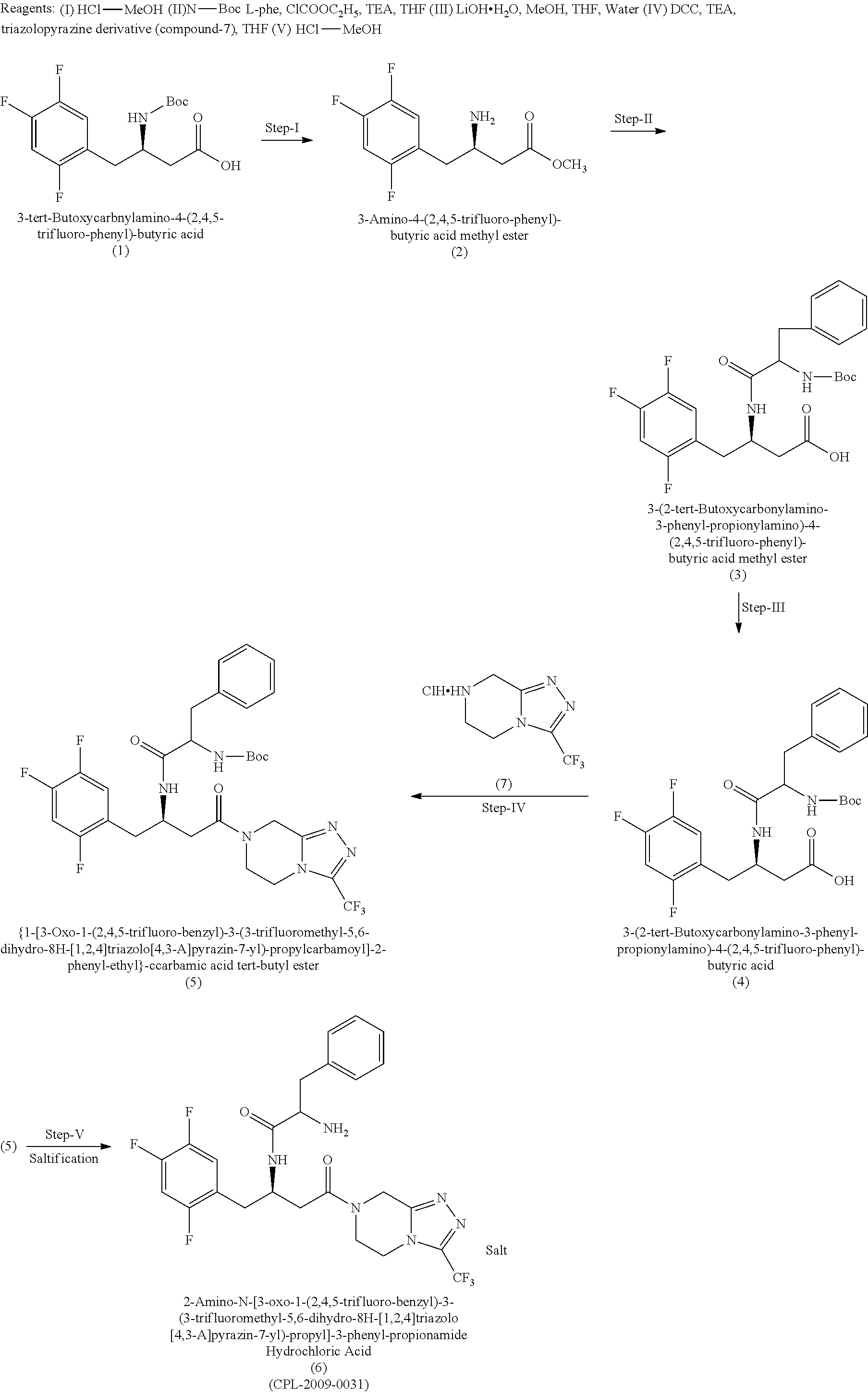 [0041] Step-I: Preparation of 3-Amino-4-(2,4,5-trifluoro-phenyl)-butyric acid methyl ester (Compound of formula 2) from 3-tert-Butoxycarbonylamino-4-(2,4,5-trifluoro-phenyl)-butyric acid (Compound of formula 1):

To a 3-tert-Butoxycarbonylamino-4-(2,4,5-trifluoro-phenyl)-butyric acid (Compound of formula 1) (12 gm, 0.036 mole), HCl-MeOH was added at room temperature and stirred for 3-4 hours. After being stirred methanol was evaporated and residue was taken into water. pH of the solution was adjusted to basic using by solid bicarbonate and DCM is added to reaction mixture. Aq. layer was extracted with DCM. The combined organic layers were dried over anhydrous Na₂SO₄, concentrated under vacuum to give 3-Amino-4-(2,4,5-trifluoro-phenyl)-butyric acid methyl ester (Compound of formula 2) as oil. (Yield=8.5 gm, 96%)
Step-II: Preparation of 3-(2-tert-Butoxycarbonylamino-3-phenyl-propionylamino)-4-(2,4,5-trifluoro-phenyl)-butyric acid methyl ester (Compound of formula 3) from 3-Amino-4-(2,4,5-trifluoro-phenyl)-butyric acid methyl ester (Compound of formula 2):

To a stirred solution of N-Boc-L-phe (6.97 gm, 0.0263 mole) in THF, triethylamine and ethylchloroformate were added at 0° C. and reaction mixture was stirred for 1 hour at room temperature. To this stirred reaction mixture, 3-Amino-4-(2,4,5-trifluoro-phenyl)-butyric acid methyl ester (Compound of formula 2) (5 gm, 0.0202 mole) was added at 0° C. and was stirred for overnight. After being stirred, THF was evaporated; residue was taken into water and aq. layer was extracted with dichloromethane. The combined organic layers were washed with bicarbonate solution and 1N HCl solution; dried over anhydrous Na₂SO₄ and concentrated to give crude. The crude was purified by silica gel column chromatography using MeOH/CH₂Cl₂ (0.2:9.8) to give 3-(2-tert-Butoxycarbonylamino-3-phenyl-propionylamino)-4-(2,4,5-trifluoro-phenyl)-butyric acid methyl ester (Compound of formula 3). (Yield=5.4 gm, 52%).
Step-III: Preparation of 3-(2-tert-Butoxycarbonylamino-3-phenyl-propionylamino)-4-(2,4,5-trifluoro-phenyl)-butyric acid (Compound of formula 4) from 3-(2-tert-Butoxycarbonylamino-3-phenyl-propionylamino)-4-(2,4,5-trifluoro-phenyl)-butyric acid methyl ester (Compound of formula 3):

To a stirred solution of 3-(2-tert-Butoxycarbonylamino-3-phenyl-propionylamino)-4-(2,4,5-trifluoro-phenyl)-butyric acid methyl ester (Compound of formula 3) (5.4 gm, 0.011 mole) in THF; MeOH, water and LiOH H₂O (0.55 gm, 0.0133 mole) were added at room temperature and stirred for 3 hours. After being stirred, solvent was evaporated and residue was taken into water. pH of the solution was adjusted to acidic using sodium hydrogen sulphate and aq. layer was extracted with dichloromethane. The combined organic layers were washed with water, dried over anhydrous Na₂SO₄ and concentrated under vacuum to give 3-(2-tert-Butoxycarbonylamino-3-phenyl-propionylamino)-4-(2,4,5-trifluoro-phenyl)-butyric acid (Compound of formula 4) as white solid. (Yield=5 gm, 96%).
Step-IV: Preparation of {1-[3-Oxo-1-(2,4,5-trifluoro-benzyl)-3-(3-trifluoromethyl-5,6-dihydro-8H-[1,2,4]triazolol[4,3-a]pyrazin-7-yl)-propylcarbamoyl]-2-phenyl-ethyl}-carbamic acid tert-butyl ester (Compound of formula 5) from 3-(2-tert-Butoxycarbonylamino-3-phenyl-propionylamino)-4-(2,4,5-trifluoro-phenyl)-butyric acid (Compound of formula 4):

To a stirred solution of 3-(2-tert-Butoxycarbonylamino-3-phenyl-propionylamino)-4-(2,4,5-trifluoro-phenyl)-butyric acid (Compound of formula 4) (5 gm, 0.0104 mole) and piperazine derivative (compound of formula 7) (2.7 gm, 0.0135 mole), HOBT was added followed by the addition of triethylamine and N,N′-dicyclohexylcarbodiimde (DCC) at 0° C. and stirred for overnight. After being stirred, dicyclohexylurea (DCU) and hydrochloride salt was removed by filtration and filtrate was concentrated under vacuum to give crude. The crude was purified by silica gel column chromatography using MeOH/CH₂Cl₂ to give {1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-2-phenyl-ethyl}-carbamic acid tert-butyl ester (Compound of formula 5). (Yield=6.0 gm, 86%).
Step-V: Preparation of 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-3-phenyl-propionamide Hydrochloric Acid (Compound of formula 6) from {1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-2-phenyl-ethyl}-carbamic acid tert-butyl ester (Compound of formula 5):

To a {1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-2-phenyl-ethyl}-carbamic acid tert-butyl ester (Compound of formula 5), HCl-MeOH was added at room temperature and stirred for 2-3 hours. After being stirred, methanol was evaporated under vacuum to give 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-3-phenyl-propionamide Hydrochloric Acid (Compound of formula 6).

### EXAMPLE-2

### Preparation of 2-Amino-3-methyl-pentanoic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of Formula 11):

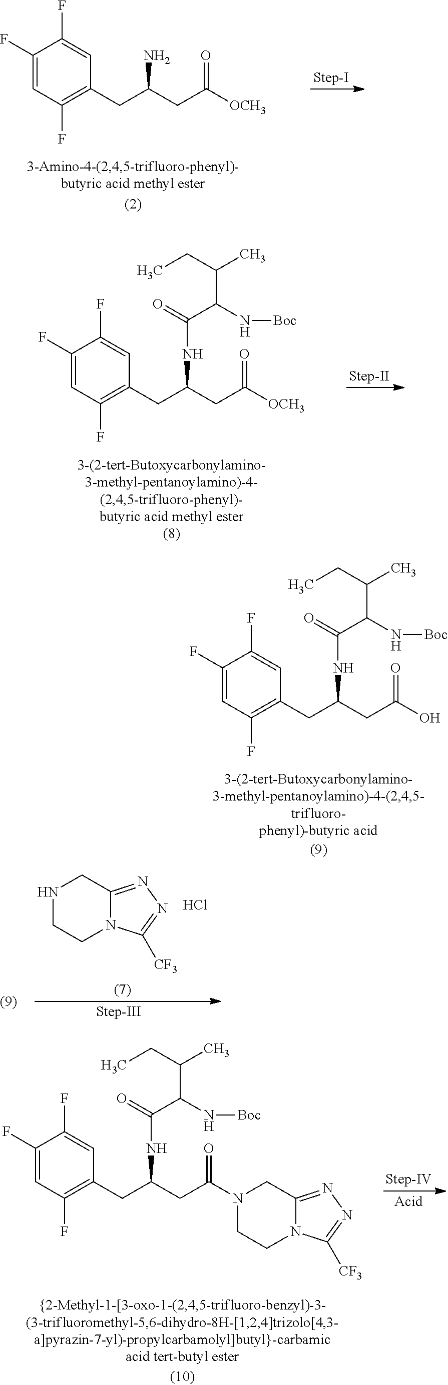 [SMILES: COC(=O)C[C@H](N)Cc1cc(F)c(F)cc1F ; CCC(C)C(NC(=O)OC(C)(C)C)C(=O)N[C@@H](CC(=O)OC)Cc1cc(F)c(F)cc1F ; CCC(C)C(NC(=O)OC(C)(C)C)C(=O)N[C@@H](CC(=O)O)Cc1cc(F)c(F)cc1F ; FC(F)(F)c1nnc2n1CCNC2 ; Cl ; CCC(C)C(NC(=O)OC(C)(C)C)C(=O)N[C@@H](CC(=O)N1CCn2c(nnc2C(F)(F)F)C1)Cc1cc(F)c(F)cc1F] 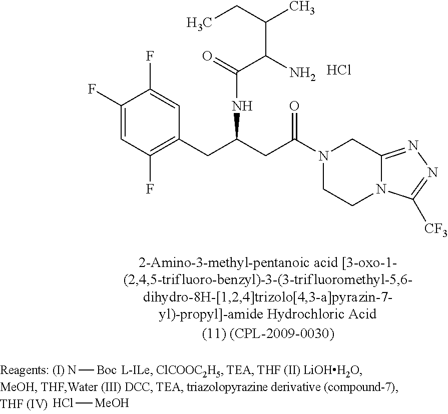 [0052] Step-I: Preparation of 3-(2-tert-Butoxycarbonylamino-3-methyl-pentanoylamino)-4-(2,4,5-trifluoro-phenyl)-butyric acid methyl ester (Compound of formula 8) from 3-Amino-4-(2,4,5-trifluoro-phenyl)-butyric acid methyl ester (Compound of formula 2):

To a stirred solution of N-Boc-L-IIe (12.34 gm, 0.0534 mole) in THF, triethylamine and ethylchloroformate were added at 0° C. and reaction mixture was stirred for 1 hour at room temperature. To this stirred reaction mixture 3-Amino-4-(2,4,5-trifluoro-phenyl)-butyric acid methyl ester (Compound of formula 2) (8 gm, 0.0202 mole) was added at 0° C. and was stirred for overnight. After being stirred THF was evaporated and residue was taken into water. Aq. layer was extracted with dichloromethane. The combined organic layers were washed with bicarbonate solution and 1N HCl solution, dried over anhydrous sodium sulphate and concentrated under vacuum to give crude. The crude was purified by silica gel column chromatography using MeOH/CH₂Cl₂ (0.2:9.8) to give 3-(2-tert-Butoxycarbonylamino-3-methyl-pentanoylamino)-4-(2,4,5-trifluoro-phenyl)-butyric acid methyl ester (Compound of formula 8). (Yield=9.7 gm, 67%).
Step-II: Preparation of 3-(2-tert-Butoxycarbonylamino-3-methyl-pentanoylamino)-4-(2,4,5-trifluoro-phenyl)-butyric acid (Compound of formula 9) from 3-(2-tert-Butoxycarbonylamino-3-methyl-pentanoylamino)-4-(2,4,5-trifluoro-phenyl)-butyric acid methyl ester (Compound of formula 8):

To a stirred solution of 3-(2-tert-Butoxycarbonylamino-3-methyl-pentanoylamino)-4-(2,4,5-trifluoro-phenyl)-butyric acid methyl ester (Compound of formula 8) (9.7 gm,0.021 mole) in THF, MeOH, water and LiOH H₂O (1.1 gm, 0.0262 mole) were added at room temperature and stirred for 3-hours. After being stirred, the solvent was evaporated, residue was taken into water, pH of the solution was adjusted to acidic with sodium hydrogen sulphate and aq. layer was extracted with dichloromethane. The combined organic layers were dried over Na₂SO₄ and concentrated under vacuum to give 3-(2-tert-Butoxycarbonylamino-3-methyl-pentanoylamino)-4-(2,4,5-trifluoro-phenyl)-butyric acid (Compound of formula 9) as white solid (Yield=9 gm, 95%).
Step-III: Preparation of {2-Methyl-1-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-butyl}-carbamic acid tert-butyl ester (Compound of formula 10) from 3-(2-tert-Butoxycarbonylamino-3-methyl-pentanoylamino)-4-(2,4,5-trifluoro-phenyl)-butyric acid (Compound of formula 9):

To a stirred solution of 3-(2-tert-Butoxycarbonylamino-3-methyl-pentanoylamino)-4-(2,4,5-trifluoro-phenyl)-butyric acid (Compound of formula 9) (6 gm, 0.0104 mole) and triazolopyrazine derivative (Compound-7) (3.2 gm, 0.0135 mole), HOBT was added followed by the addition of triethylamine and N,N′-dicylohexyldicarbodiimide (DCC) at 0° C. and stirred for overnight. After being stirred dicyclohexylurea (DCU), hydrochloride salt was removed by filtration and filtrate was concentrated under vacuum to give crude. The crude was purified by silica gel column chromatography using MeOH/CH₂Cl₂ to give {2-Methyl-1-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-butyl}-carbamic acid tert-butyl ester (Compound of formula 10). (Yield=5 gm, 60%).
Step-IV: Preparation of 2-Amino-3-methyl-pentanoic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of formula 11) from {2-Methyl-1-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-butyl}-carbamic acid tert-butyl ester (Compound of formula 10):

To a {2-Methyl-1-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-butyl}-carbamic acid tert-butyl ester (Compound of formula 10), HCl-MeOH was added at room temperature and stirred for 2-3 hours. After being stirred, methanol was evaporated under vacuum to give 2-Amino-3-methyl-pentanoic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of formula 11).

### EXAMPLE-3

### Preparation of 1-(2-Amino-3-methyl-butyryl)-pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of Formula 15):

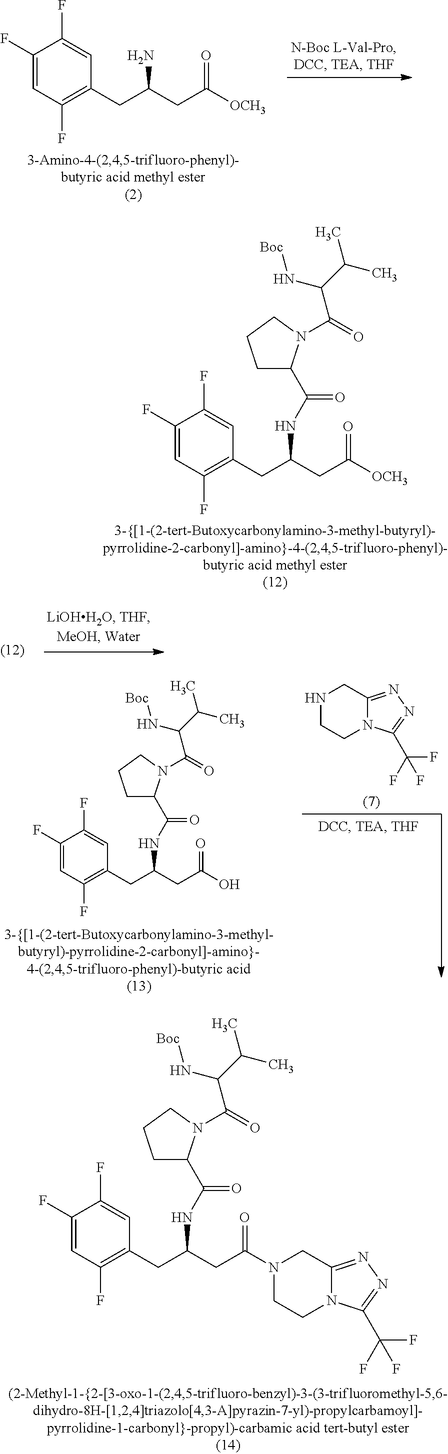 [SMILES: COC(=O)C[C@H](N)Cc1cc(F)c(F)cc1F ; COC(=O)C[C@@H](Cc1cc(F)c(F)cc1F)NC(=O)C1CCCN1C(=O)C(NC(=O)OC(C)(C)C)C(C)C ; CC(C)C(NC(=O)OC(C)(C)C)C(=O)N1CCCC1C(=O)N[C@@H](CC(=O)O)Cc1cc(F)c(F)cc1F ; FC(F)(F)c1nnc2n1CCNC2 ; CC(C)C(NC(=O)OC(C)(C)C)C(=O)N1CCCC1C(=O)N[C@@H](CC(=O)N1CCn2c(nnc2C(F)(F)F)C1)Cc1cc(F)c(F)cc1F] 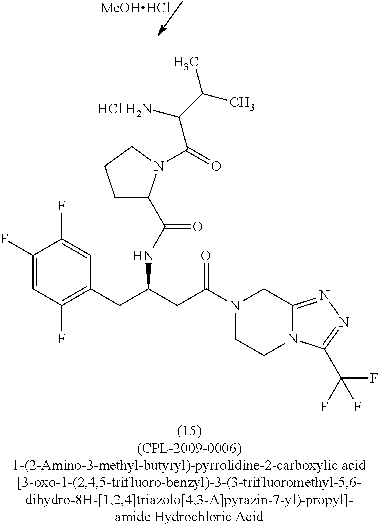 [0061] Step-I: Preparation of 3-{[1-(2-tert-Butoxycarbonylamino-3-methyl-butyryl)-pyrrolidine-2-carbonyl]-amino}-4-(2,4,5-trifluoro-phenyl)-butyric acid methyl ester (Compound of formula 12) from 3-Amino-4-(2,4,5-trifluoro-phenyl)-butyric acid methyl ester (Compound of formula 2):

To a stirred solution of N-Boc-L-Val-Pro (1.2 gm, 0.0038 mole) in THF, triethylamine and ethylchloroformate were added at 0° C. and reaction mixture was stirred for 1 hour at room temperature. To this stirred reaction mixture, 3-Amino-4-(2,4,5-trifluoro-phenyl)-butyric acid methyl ester (Compound of formula 2) (1.3 gm, 0.0052 mole) was added at 0° C. and stirred for overnight. After being stirred, THF was evaporated, residue was taken into water and aq. layer was extracted with dichloromethane. The combined organic layers were washed with bicarbonate solution and 1N HCl solution, dried over anhydrous sodium sulphate and concentrated under vacuum to give crude. The crude was purified by silica gel column chromatography using MeOH/CH₂Cl₂ (0.2:9.8) to give 3-{[1-(2-tert-Butoxycarbonylamino-3-methyl-butyryl)-pyrrolidine-2-carbonyl]-amino}-4-(2,4,5-trifluoro-phenyl)-butyric acid methyl ester (Compound of formula 12) (Yield=0.8 gm, 27%).
Step-II: Preparation of 3-{[1-(2-tert-Butoxycarbonylamino-3-methyl-butyryl)-pyrrolidine-2-carbonyl]-amino}-4-(2,4,5-trifluoro-phenyl)-butyric acid (Compound of formula 13) from 3-{[1-(2-tert-Butoxycarbonylamino-3-methyl-butyryl)-pyrrolidine-2-carbonyl]-amino}-4-(2,4,5-trifluoro-phenyl)-butyric acid methyl ester (Compound of formula 12):

To a stirred solution of 3-{[1-(2-tert-Butoxycarbonylamino-3-methyl-butyryl)-pyrrolidine-2-carbonyl]-amino}-4-(2,4,5-trifluoro-phenyl)-butyric acid methyl ester (Compound of formula 12) (0.780 gm, 0.0014 mole) in THF, MeOH, water and LiOH.H₂O (0.066 gm,0.0016 mole) were added at room temperature and stirred for 3-hours. After being stirred, the solvent was evaporated, residue was taken into water, pH of the solution was adjusted to acidic with sodium hydrogen sulphate and aq. layer was extracted with dichloromethane. The combined organic layers were dried over Na₂SO₄ and concentrated under vacuum to give 3-{[1-(2-tert-Butoxycarbonylamino-3-methyl-butyryl)-pyrrolidine-2-carbonyl]-amino}-4-(2,4,5-trifluoro-phenyl)-butyric acid (Compound of formula 13) as white solid (Yield=0.674 gm, 93.6%).
Step-III: Preparation of (2-Methyl-1-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidine-1-carbonyl}-propyl)-carbamic acid tert-butyl ester (Compound of formula 14) from 3-{[1-(2-tert-Butoxycarbonylamino-3-methyl-butyryl)-pyrrolidine-2-carbonyl]-amino}-4-(2,4,5-trifluoro-phenyl)-butyric acid (Compound of formula 13):

To a stirred solution of 3-{[1-(2-tert-Butoxycarbonylamino-3-methyl-butyryl)-pyrrolidine-2-carbonyl]-amino}-4-(2,4,5-trifluoro-phenyl)-butyric acid (Compound of formula 13) (0.650, 0.0013 mole) and piperazine derivative (compound-7) (0.320 gm, 0.0014 mole), HOBT was added followed by the addition of triethylamine and N, N′-dicyclohexylcarbodiimide (DCC) at 0° C. and stirred for overnight. After being stirred, dicyclohexylurea (DCU) and hydrochloride salt were removed by filtration and the filtrate was concentrated under vacuum to give crude. The crude was purified by silica gel column chromatography using MeOH/CH₂Cl₂ to give (2-Methyl-1-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidine-1-carbonyl}-propyl)-carbamic acid tert-butyl ester (Compound of formula 14) (Yield=0.504 gm, 58.60%).
Step-IV: Preparation of 1-(2-Amino-3-methyl-butyryl)-pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of formula 15) from (2-Methyl-1-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidine-1-carbonyl}-propyl)-carbamic acid tert-butyl ester (Compound of formula 14):

To the (2-Methyl-1-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidine-1-carbonyl}-propyl)-carbamic acid tert-butyl ester (Compound of formula 14), HCl-MeOH was added at room temperature and stirred for 2-3 hours. After being stirred, methanol was evaporated under vacuum to give of 1-(2-Amino-3-methyl-butyryl)-pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of formula 15).

### EXAMPLE-4

### Preparation of 2-Amino-N-[3-(2-cyano-pyrolidin-1-yl)-3-oxo-1-(2,4,5-trifluoro-benzyl)-propyl]-3-phenyl-propionamide trifluoro-acetic acid (Compound of Formula 18):

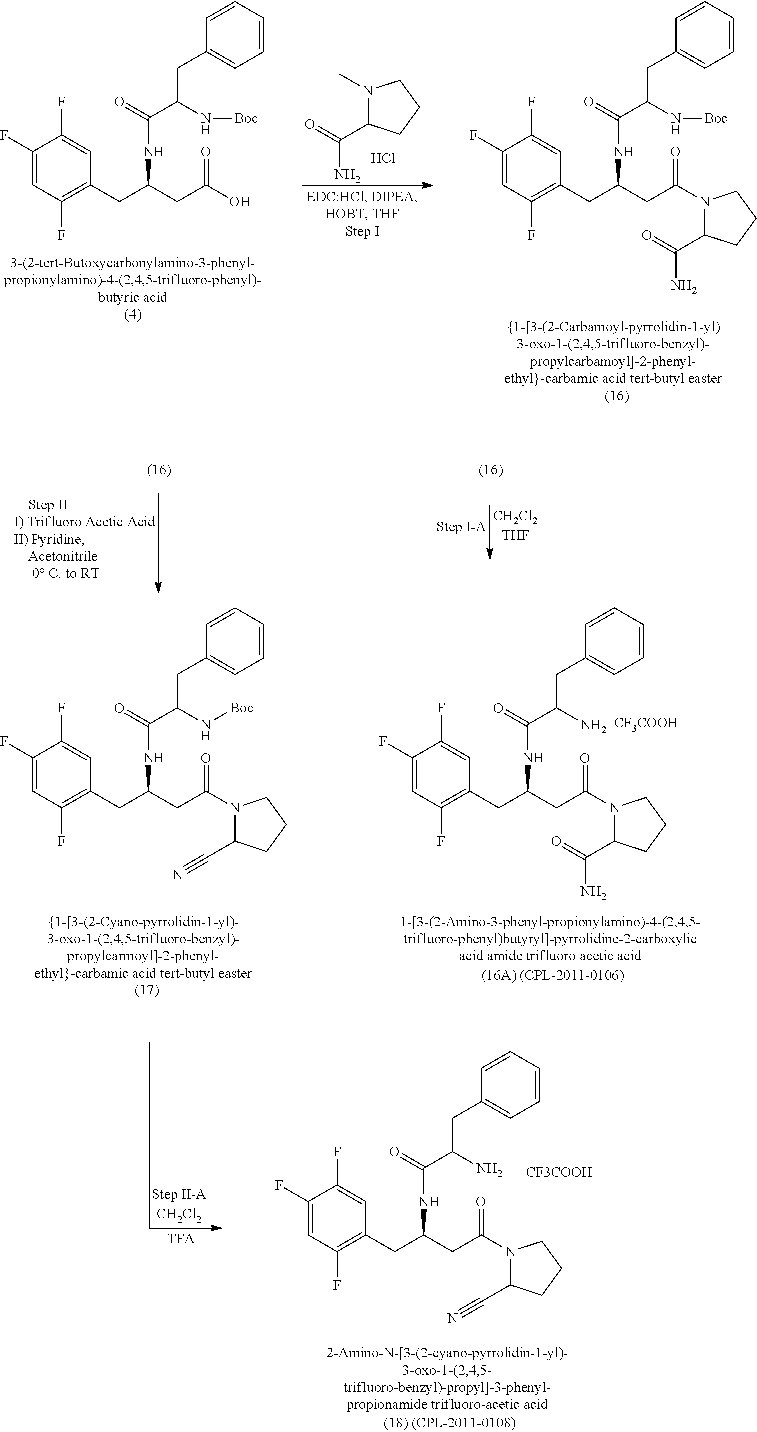 [0070] Step I: Preparation of {1-[3-(2-Carbamoyl-pyrrolidin-1-yl)-3-oxo-1-(2,4,5-trifluoro-benzyl)propylcarbamoyl]-2-phenyl-ethyl}-carbamic acid tert-butvl ester (Compound of formula 16) from 3-(2-tert-Butoxycarbonylamino-3-phenyl-propionylamino)-4-(2,4,5-trifluoro-phenyl)-butyric acid (Compound of formula 4):

To a stirred solution of 3-(2-tert-Butoxycarbonylamino-3-phenyl-propionylamino)-4-(2,4,5-trifluoro-phenyl)-butyric acid (Compound of formula 4) (2.52 gm, 0.0052 mole), N, N-Diisopropylethylamine (DIPEA) (1.26 ml, 0.00781 mol) in THF and 1-hydroxybenztriazole (0.95 gm, 0.00624 mol) was added at 0° C. and stirred at room temperature for 1 hour. To the stirred solution, EDC:HCl (1.29 gm, 0.00676 mol) and tri-fluoro acetic acid salt of prolinamide (1.2 gm, 0.00520 mol) were added at 0° C. and stirred at room temperature overnight. After being stirred, the reaction mass was concentrated over the rotary evaporator, saturated aq. NaHCO₃ was added and filtered to give crude. The crude was washed with 1N HCl followed by washed by hexane and dried to give pure {1-[3-(2-Carbamoyl-pyrrolidin-1-yl)-3-oxo-1-(2,4,5-trifluoro-benzyl)-propylcarbamoyl]-2-phenyl-ethyl}-carbamic acid tert-butyl ester (Compound of formula 16). (Yield=1.80 gm, 60%)
Step I-A: Preparation of 1-[3-(2-Amino-3-phenyl-propionylamino)-4-(2,4,5-trifluoro-phenyl)-butyryl]-pyrrolidine-2-carboxylic acid amide trifluoro acetic acid (Compound of formula 16-A) from {1-[3-(2-Carbamoyl-pyrrolidin-1-yl)-3-oxo-1-(2,4,5-trifluoro-benzyl)propylcarbamoyl]-2-phenyl-ethyl}-carbamic acid tert-butvl ester (Compound of formula 16):

To the solution of {1-[3-(2-Carbamoyl-pyrrolidin-1-yl)-3-oxo-1-(2,4,5-trifluoro-benzyl)-propylcarbamoyl]-2-phenyl-ethyl}-carbamic acid tert-butyl ester (Compound of formula 16) (500 mg) in dichloromethane (10 ml), Tri-fluoro acetic acid (1 ml)) was added at 0° C. and stirred for 1 hours at room temperature. After being stirred, the reaction mixture was concentrated over rota vapour to give crude. The crude was dried under the vacuum and purified to give 1-[3-(2-Amino-3-phenyl-propionylamino)-4-(2,4,5-trifluoro-phenyl)-butyryl]-pyrrolidine-2-carboxylic acid amide trifluoro acetic acid (Compound of formula 16-A). (Yield=350 mg, 68%).
Step II: Preparation of {1-[3-(2-Cyano-pyrrolidin-1-yl)-3-oxo-1-(2,4,5-trifluoro-benzyl)-propylcarbamoyl]-2-phenyl-ethyl}-carbamic acid tert-butvl ester (Compound of formula 17) from {1-[3-(2-Carbamoyl-pyrrolidin-1-yl)-3-oxo-1-(2,4,5-trifluoro-benzyl)-propylcarbamoyl]-2-phenyl-ethyl}-carbamic acid tert-butyl ester (Compound of formula 16):

To the solution of dichloromethan (30 ml) and pyridine (1 ml), {1-[3-(2-Carbamoyl-pyrrolidin-1-yl)-3-oxo-1-(2,4,5-trifluoro-benzyl)-propylcarbamoyl]-2-phenyl-ethyl}-carbamic acid tert-butyl ester (Compound of formula 16) (1 gm, 0.00173 mol) was added at 0° C. followed by the addition of tri-fluoro acetic anhydride (0.6 ml, 0.00432 mol) drop-wise at 0° C. and stirred for 3-5 hours. The mixture of toluene (5 ml) and ethyl acetate (10 ml) was added and stirred followed by concentrated to give crude. The crude was purified by column chromatography to give {1-[3-(2-Cyano-pyrrolidin-1-yl)-3-oxo-1-(2,4,5-trifluoro-benzyl)-propylcarbamoyl]-2-phenyl-ethyl}-carbamic acid tert-butyl ester (Compound of formula 17). (Yield=157 mg, 16%)
Step II-A: Preparation of 2-Amino-N-[3-(2-cyano-pyrrolidin-1-yl)-3-oxo-1-(2,4,5-trifluoro-benzyl)-propyl]-3-phenyl-propionamide trifluoro-acetic acid (Compound of formula 18) from {1-[3-(2-Cyano-pyrrolidin-1-yl)-3-oxo-1-(2,4,5-trifluoro-benzyl)-propylcaramoyl]-2-phenyl-ethyl}-carbamic acid tert-butyl ester (Compound of formula 17):

To the solution of {1-[3-(2-Cyano-pyrrolidin-1-yl)-3-oxo-1-(2,4,5-trifluoro-benzyl)-propylcarbamoyl]-2-phenyl-ethyl}-carbamic acid tert-butyl ester (Compound of formula 17) (700 mg) in dichloromethane (15 ml), Tri-fluoro acetic acid (2 ml)) was added at 0° C. and stirred for 1 hours at room temperature. After being stirred, the reaction mixture was concentrated over rota vapour to give crude. The crude was dried under the vacuum and purified to give 2-Amino-N-[3-(2-cyano-pyrrolidin-1-yl)-3-oxo-1-(2,4,5-trifluoro-benzyl)-propyl]-3-phenyl-propionamide trifluoro-acetic acid (Compound of formula 18). (Yield=205 mg, 29%).

EXAMPLES-5

### Preparation of Pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of Formula 21):

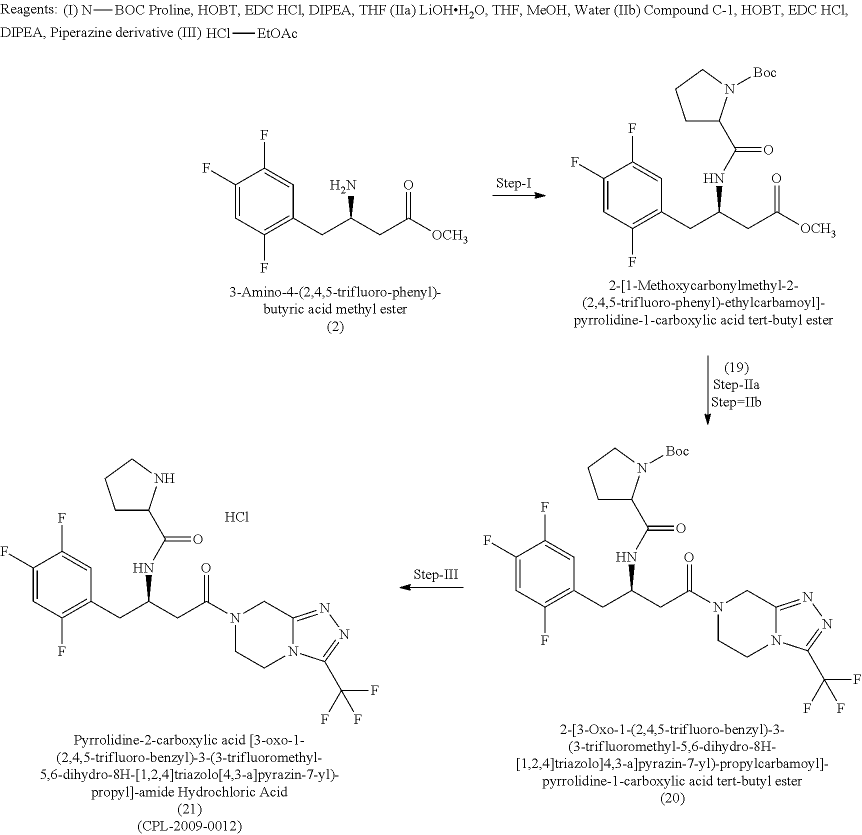 [0080] Step-I: Preparation of 2-[1-Methoxycarbonylmethyl-2-(2,4,5-trifluoro-phenyl)-ethylcarbamoyl]-pyrrolidine-1-carboxylic acid tert-butyl ester (Compound of formula 19) from 3-Amino-4-(2,4,5-trifluoro-phenyl)-butyric acid methyl ester (Compound of formula 2):

To a stirred solution of N-Boc-Proline (2.6 gm, 0.0121 mole), 1-hydroxybenztriazole (2.2 gm, 0.0145 mole) and N, N′-diisopropylethylethylamine (2.6 gm, 0.0204 mole) in THF (30 ml), the mixture of EDC.HCl (3.0 gm, 0.0157 mole) and 3-Amino-4-(2,4,5-trifluoro-phenyl)-butyric acid methyl ester (Compound of formula 2) (3 gm, 0.0121 mole) were added at 0° C. The reaction mixture was then stirred at room temperature for overnight. After being stirred, THF was evaporated and residue was taken into water. Aq. layer was extracted with ethyl acetate (120 ml). The combined organic layers were washed with 1N HCl and aq. bicarbonate followed by drying over sodium sulphate and concentrated under vacuum to give 2-[1-Methoxycarbonylmethyl-2-(2,4,5-trifluoro-phenyl)-ethylcarbamoyl]-pyrrolidine-1-carboxylic acid tert-butyl ester (Compound of formula 19) as white solid (Yield=4.3 gms, 81%).
Step II: Preparation of 2-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidine-1-carboxylic acid tert-butyl ester (Compound of formula 20) 2-[1-Methoxycarbonylmethyl-2-(2,4,5-trifluoro-phenyl)-ethylcarbamoyl]-pyrrolidine-1-carboxylic acid tert-butyl ester (Compound of formula 19):

To a stirred solution of 2-[1-Methoxycarbonylmethyl-2-(2,4,5-trifluoro-phenyl)-ethylcarbamoyl]-pyrrolidine-1-carboxylic acid tert-butyl ester (Compound of formula 19) (4.3 gms, 0.0096 mole) in THF (20 ml); methanol (6 ml), water (6 ml) and LiOH.H₂O (0.487 gm, 0.0116 mole) were added at room temperature and reaction mixture was stirred for 3 hours. After being stirred, solvent was evaporated and resulting residue was taken into water. The aq. layer was acidified with 1N HCl and extracted with ethyl acetate (140 ml). The combined organic layers were dried over sodium sulphate and concentrated in vacuum to give compound of formula 19a as white solid (Yield=4.0 gm, 97%).

To this stirred solution of compound of formula 19a (4.0 gm, 0.009 mole), 1-hydroxybenztriazole (1.7 g, 0.0111 mole), piperazine derivative (2.3 gm, 0.0102 mole) and N,N-diisopropylethylamine (2.6 gm, 0.0204 mole) in THF, EDC.HCl (2.3 gm, 0.0120 mole) and N,N-diisopropylethylamine (2.0 gm, 0.0156 mole) were added at 0° C. and reaction mixture was stirred for overnight at room temperature. After being stirred, THF was evaporated and residue was taken into water. The aq. layer was extracted with ethyl acetate (150 ml). The combined organic layers were washed with 1N HCl and aq. bicarbonate followed by dried over sodium sulphate and concentrated in vacuum to give crude. The crude was purified by column chromatography to give 2-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidine-1-carboxylic acid tert-butyl ester (Compound of formula 20) as off-white solid. (Yield=4.9 gm, 87%).
Step III: Preparation of Pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide Hydrochloric Acid (Compound of Formula 21) from 2-[3-Oxo-1-(2,4,5-trifluoro-benzY1)-3-(3-trifluoromethyl-5,6-dihydro-8H-[1,2,4]triazolo[4,3-a]pyrazin-7-yl)-propylcarbamoyl]-pyrrolidine-1-carboxylic acid tert-butyl ester (Compound of formula 20):

To the 2-[3-Oxo-1-(2,4,5-trifl uoro-benzyl)-3-(3-trifluoromethyl-5,6-dihydro-8H-[1,2,4]triazolo[4,3-a]pyrazin-7-yl)-propylcarbamoyl]-pyrrolidine-1-carboxylic acid tert-butyl ester (Compound of formula 20) (4.0 gm, 0.0066 mole), hydrochloride solution in ethyl acetate was added at room temperature and stirred for 2 hours. After being stirred, ethyl acetate was evaporated in vacuum and resulting oily material was triturated with diisopropylether to give Pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]amide Hydrochloric Acid (Compound of Formula 21) as white solid (Yield=3.3 gm, 94%).

### EXAMPLE 6

### Preparation of 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-propionamide Hydrochloric Acid (Compound of Formula 24):

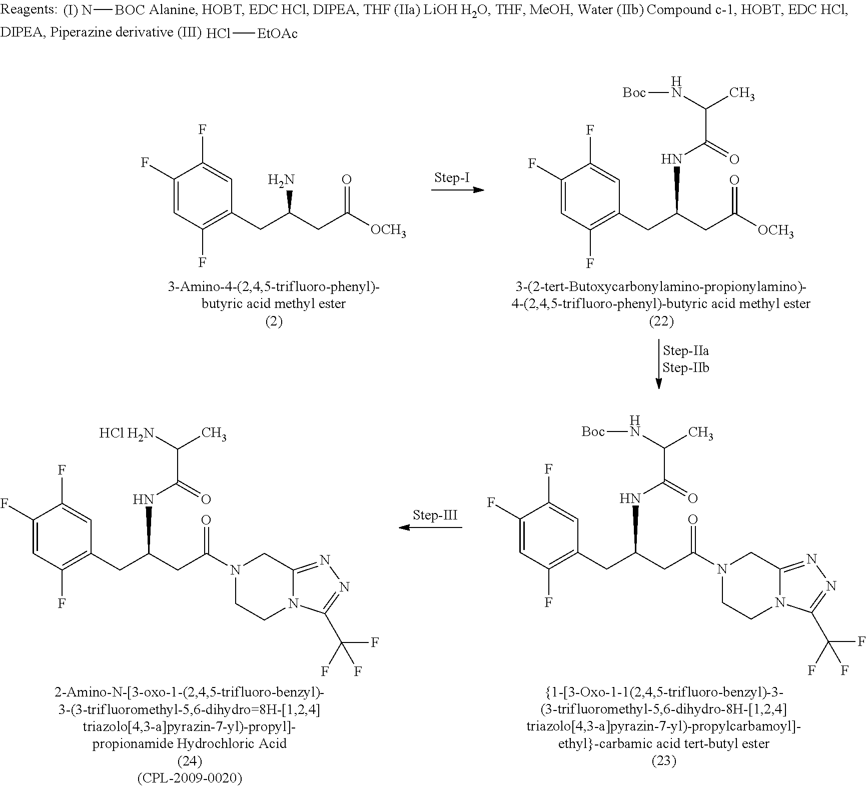 [0088] Step-I: Preparation of 3-(2-tert-Butoxycarbonylamino-propionylamino)-4-(2,4,5-trifluoro-phenyl)-butyric acid methyl ester (Compound of formula 22) from 3-Amino-4-(2,4,5-trifluoro-phenyl)-butyric acid methyl ester (Compound of formula 2):

To a stirred solution of N-BOC-Alanine (2.2 gm, 0.0121 mole), 1-hydroxybenztriazole (2.2 gm, 0.0145 mole) and N,N-diisopropylethylethylamine (2.6 gm, 0.0204 mole) in THF (30 ml), EDC.HCl (3.0 gm, 0.0157 mole) and 3-Amino-4-(2,4,5-trifluoro-phenyl)-butyric acid methyl ester (Compound of formula 2) (3 gm, 0.0121 mole) were added at 0° C. The reaction mixture was then stirred at room temperature for overnight. After being stirred, THF was evaporated and residue was taken into water. It was then extracted with ethyl acetate (120 ml). The combined organic layers were washed with 1N HCl and aq. bicarbonate followed by dried over sodium sulphate and concentrated in vacuum to give 3-(2-tert-Butoxycarbonylamino-propionylamino)-4-(2,4,5-trifluoro-phenyl)-butyric acid methyl ester (Compound of formula 22) as white solid (Yield=4.7 gm, 94%).
Step-II: Preparation of {1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-ethyl}-carbamic acid tert-butyl ester (Compound of formula 23) from 3-(2-tert-Butoxycarbonylamino-propionylamino)-4-(2,4,5-trifluoro-phenyl)-butyric acid methyl ester (Compound of formula 22):

To a stirred solution of 3-(2-tert-Butoxycarbonylamino-propionylamino)-4-(2,4,5-trifluoro-phenyl)-butyric acid methyl ester (Compound of formula 22) (4.7 gm, 0.0112 mole) in THF (20 ml), methanol (6 ml), water (6 ml) and LiOH.H₂O (0.566 gm, 0.0134 mole) were added at room temperature and reaction mixture was stirred for 3 hours. After being stirred, solvent was evaporated and resulting residue was taken into water. Aq. layer was acidified with 1N HCl and extracted with ethyl acetate (140 ml). The combined organic layers were dried over sodium sulphate and concentrated in vacuum to give compound of formula 22a as white solid. (Yield=4.5 gm, 97%).

To this stirred solution of compound of formula 22a (4.5 gm, 0.011 mole), 1-hydroxybenztriazole (2.0g, 0.0133 mole), piperazine derivative (2.7 gm, 0.0122 mole) and N,N-diisopropylethylamine (3.1 gm, 0.0244 mole) in THF, EDC.HCl (2.7 gm, 0.0144 mole) and N,N-diisopropylethylamine (2.4 gm, 0.0187 mole) were added at 0° C. and reaction mixture was stirred for overnight at room temperature. After being stirred, THF was evaporated and residue was taken into water. The aq. layer was extracted with ethyl acetate (150 ml). The combined organic layers were washed with 1N HCl and aq. bicarbonate, dried over sodium sulphate and concentrated in vacuum to give crude. The crude was purified by column chromatography to give {1-[3-Oxo-1-(2,4,5-trifluoro-benzyl)-3-(3-trifluoromethyl-5,6-dihydro-8H -[1,2,4]triazolo[4,3-a]pyrazin-7-yl)-propylcarbamoyl]-ethyl}-carbamic acid tert-butyl ester (Compound of formula 23) as off-white solid. (Yield=5.3 gm, 82%).
Step-C: Preparation of 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-propionamide Hydrochloric Acid (Compound of formula 24) from {1-[3-Oxo-1-(2,4,5-trifluoro-benzyl-3-(3-trifluoromethyl-5,6-dihydro-8H-[1,2,4]triazolo[4,3-a]pyrazin-7-14)-propylcarbamoyl]-ethyl}-carbamic acid tert-butyl ester (Compound of formula 23:

To this {1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]ethyl}-carbamic acid tert-butyl ester (Compound of formula 23) (4.0 gm, 0.0069 mole), hydrochloride solution in ethyl acetate was added at room temperature and stirred for 2 hours. After being stirred, ethyl acetate was evaporated in vacuum and resulting oily material was triturated with diisopropylether to give 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-propionamide Hydrochloric Acid (Compound of formula 24) as white solid. (Yield=3.2 gm, 91%).

### EXAMPLE 7

### Preparation of 1-(2-Amino-3-phenyl-propionyl)-pyrrolidine-2-carboxylicacid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide hydrochloric acid (Compound of formula 32):

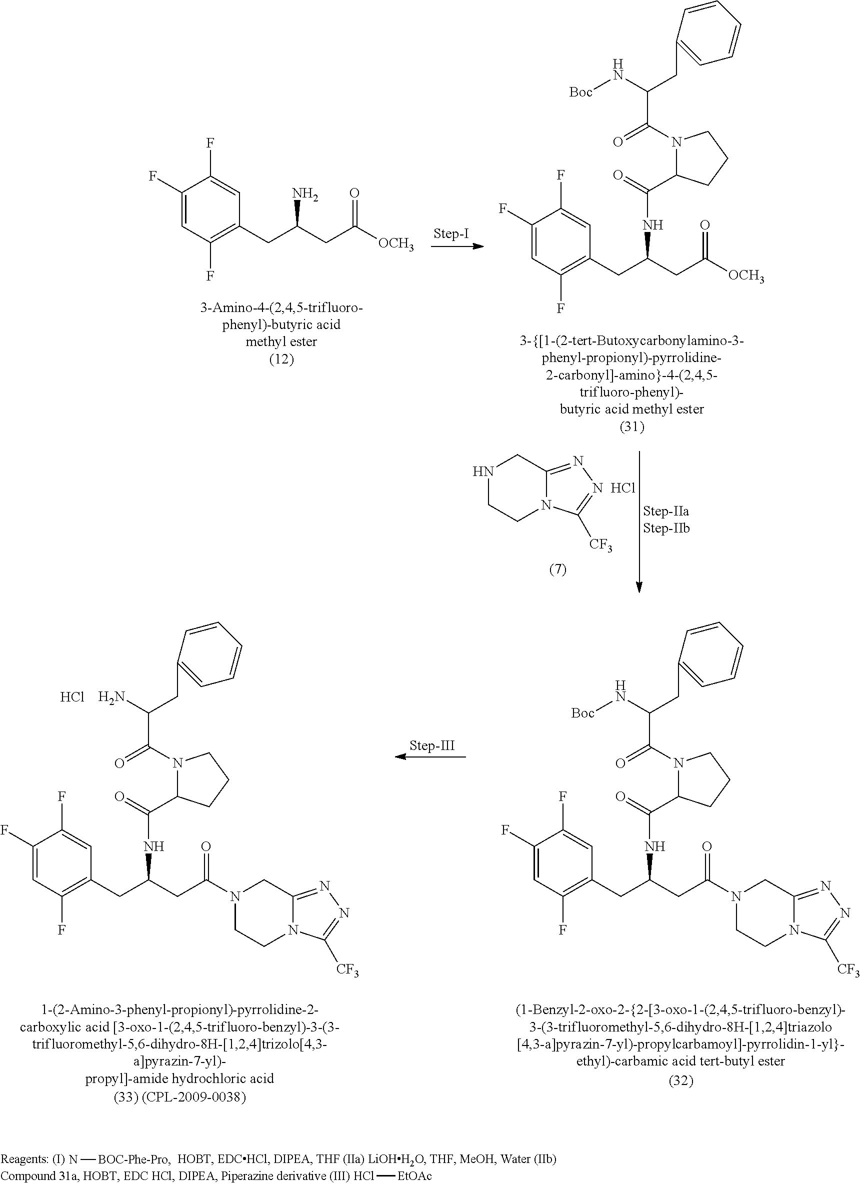 [0096] STEP-I: Preparation of 3-{[1-(2-tert-Butoxycarbonylamino-3-phenyl-propionyl)-pyrrolidine-2-carbonyl]-amino}-4-(2,4,5-trifluoro-phenyl)-butyric acid methyl ester (Compound of formula 31) from 3-Amino-4-(2,4,5-trifluoro-phenyl)-butyric acid methyl ester (Compound of formula 2):

To a stirred solution of N-Boc-Phe-Pro (1.0 gm, 0.0027 mole), 1-hydroxybenztriazole (0.496 gm, 0.0032 mole) and N,N-diisopropylethylethylamine (0.58 gm, 0.0045 mole) in THF (30 ml), EDC.HCl (0.672 gm, 0.0035 mole) and 3-Amino-4-(2,4,5-trifluoro-phenyl)-butyric acid methyl ester (Compound of formula 2) (0.68, 0.0027 mole) were added at 0° C. The reaction mixture was then stirred at room temperature for overnight. After being stirred, THF was evaporated and residue was taken into water. It was then extracted with ethyl acetate (70 ml). The combined organic layers were washed with 1N HCl and aq. bicarbonate, dried over sodium sulphate and concentrated in vacuum to give 3-{[1-(2-tert-Butoxycarbonylamino-3-phenyl-propionyl)-pyrrolidine-2-carbonyl]-amino}-4-(2,4,5-trifluoro-phenyl)-butyric acid methyl ester (Compound of formula 31) as white solid (Yield=1.5 gm, 93%).
STEP-II: Preparation of (1-Benzyl-2-oxo-2-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidin-1-yl}-ethyl)-carbamic acid tert-butyl ester (Compound of formula 32) from 3-{[1-(2-tert-Butoxycarbonylamino-3-phenyl-propionyl)-pyrrolidine-2-carbonyl]-amino}-4-(2,4,5-trifluoro-phenyl)-butyric acid methyl ester (Compound of formula 31):

To a stirred solution of 3-{[1-(2-tert-Butoxycarbonylamino-3-phenyl-propionyl)-pyrrolidine-2-carbonyl]-amino}-4-(2,4,5-trifluoro-phenyl)-butyric acid methyl ester (Compound of formula 31) (1.5 gm, 0.0025 mole) in THF (10 ml), methanol (3 ml), water (3 ml) and LiOH.H₂O (0.127 gm, 0.0030 mole) were added at room temperature and reaction mixture was stirred for 3 hours. After being stirred, solvent was evaporated and resulting residue was taken into water. The aq. layer was acidified with 1N HCl and extracted with ethyl acetate (60 ml). The combined organic layers were dried over sodium sulphate and concentrated in vacuum to give compound of formula 31-a as white solid. (Yield=1.0 gm, 68%).

To this stirred solution of compound of formula 31-a (1.0 gm, 0.0017 mole), 1-hydroxybenztriazole (0.26 gm, 0.0017 mole), piperazine derivative (0.407 gm, 0.0017 mole) and N,N-diisopropylethylamine (0.37 gm, 0.00287 mole) in THF (25 ml), EDC.HCl (0.42 gm, 0.0022 mole) and N,N-diisopropylethylamine (0.460 gm, 0.00356 mole) were added at 0° C. and reaction mixture was stirred for overnight at room temperature. After being stirred, THF was evaporated and residue was taken into water. The aq. layer was extracted with ethyl acetate (60 ml). The combined organic layers were washed with 1N HCl and aq. bicarbonate, dried over sodium sulphate and concentrated in vacuum to give crude. The crude was purified by column chromatography to give (1-Benzyl-2-oxo-2-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidin-1-yl}-ethyl)-carbamic acid tert-butyl ester (Compound of formula 32) as off-white solid. (Yield=0.7 gm, 50%).
STEP-III: Preparation of 1-(2-Amino-3-phenyl-propionyl)-pyrrolidine-2-carboxylicacid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide hydrochloric acid (Compound of formula 33) from (1-Benzyl-2-oxo-2-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidin-1-yl}-ethyl)-carbamic acid tert-butyl ester (Compound of formula 32):

To this (1-Benzyl-2-oxo-2-{2-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-pyrrolidin-1-yl}-ethyl)-carbamic acid tert-butyl ester (Compound of formula 32) (0.5 gm, 0.00066 mole), hydrochloride solution in ethyl acetate was added at room temperature and stirred for 2 hours. After being stirred ethyl acetate was evaporated in vacuum and resulting oily material was triturated with diisopropylether to give 1-(2-Amino-3-phenyl-propionyl)-pyrrolidine-2carboxylic acid 1-(2-Amino-3-phenyl-propionyl)-pyrrolidine-2-carboxylicacid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide hydrochloric acid (Compound of formula 33) as white solid (Yield=0.4 gm, 87%).

### EXAMPLE-8

### Preparation of 2-Amino-N-{1-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-ethyl}-3-phenyl-propionamide (Compound of Formula 39):

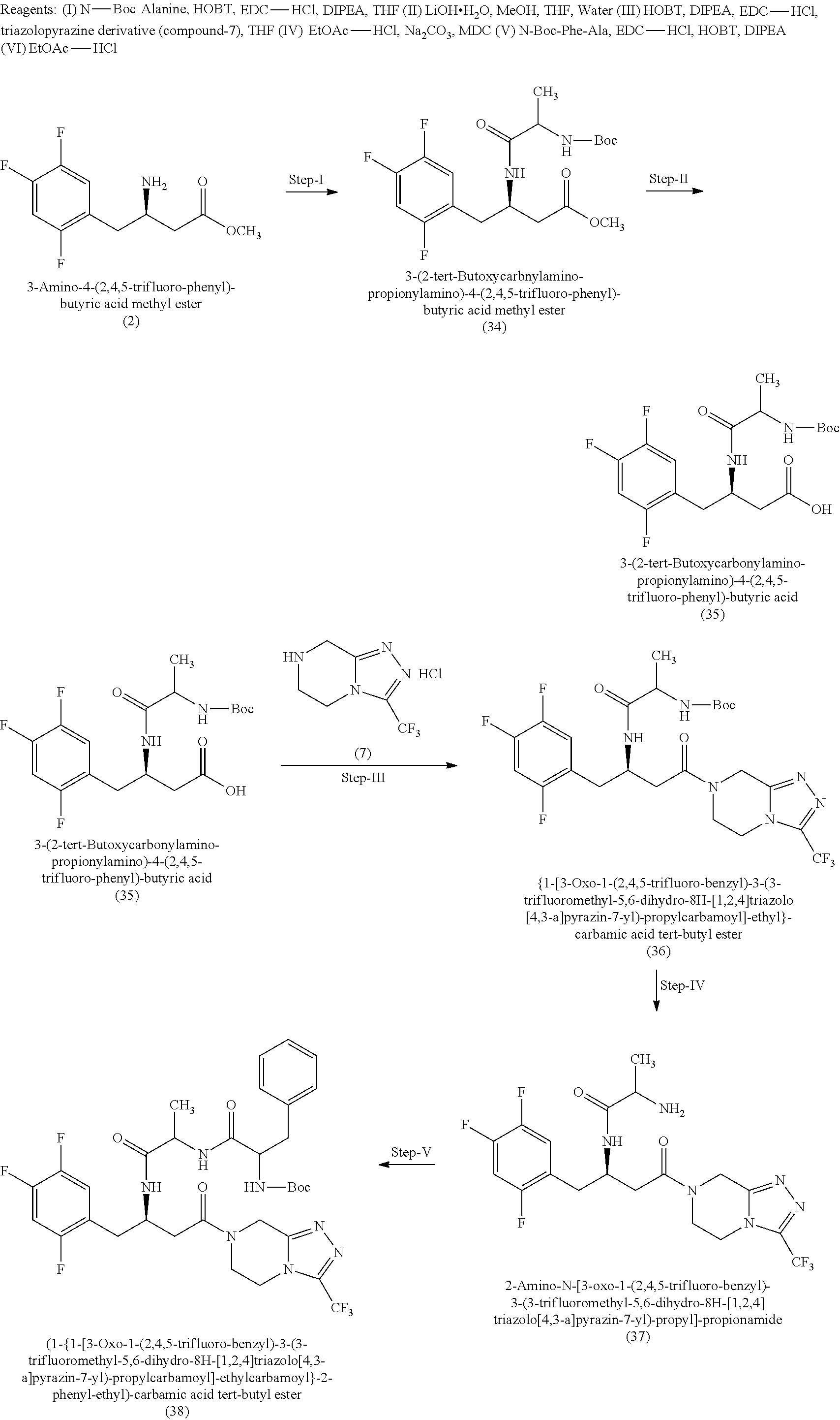 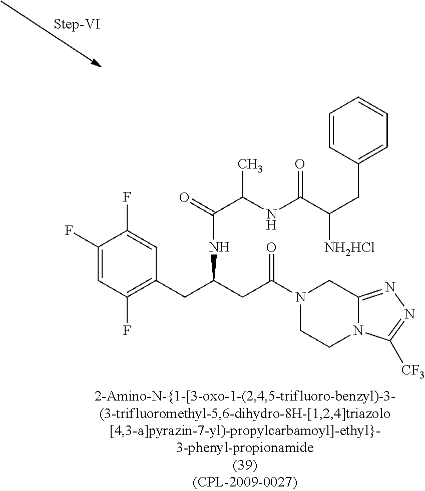 [0104] Step-I: Preparation of 3-(2-tert-Butoxycarbonylamino-propionylamino)-4-(2,4,5-trifluoro-phenyl)-butyric acid methyl ester (Compound of formula 34) from 3-Amino-4-(2,4,5-trifluoro-phenyl)-butyric acid methyl ester (Compound of formula 2):

To a stirred solution of N-Boc-Alanine (2.2 gm, 0.0121 mol) in THF, hydroxyl benztriazole and N,N-diisopropyl ethyl amine were added at 0° C. and reaction mixture was stirred at room temperature. To this stirred reaction mixture, 3-Amino-4-(2,4,5-trifluoro-phenyl)-butyric acid methyl ester (Compound of formula 2) (3 gm, 0.0121 mol) and EDC-HCl were added at 0° C. and was stirred for overnight at room temperature. After being stirred, THF was evaporated and residue was taken into saturated aq. NaHCO₃ solution (50 ml). Aq. layer was extracted with dichloromethane. The combined organic layers were washed with 1N HCl, dried over anhydrous sodium sulphate and concentrated under vacuum to give crude. The crude was purified by crystallization to give 3-(2-tert-Butoxycarbonylamino-propionylamino)-4-(2,4,5-trifluoro-phenyl)-butyric acid methyl ester (Compound of formula 34). (Yield=4.7 gm, 94%).
Step-II: Preparation of 3-(2-tert-Butoxycarbonylamino-propionylamino)-4-(2,4,5-trifluoro-phenyl)-butyric acid (Compound of formula 35) from 3-(2-tert-Butoxycarbonylamino-propionylamino)-4-(2,4,5-trifluoro-phenyl)-butyric acid methyl ester (Compound of formula 34):

To a stirred solution of 3-(2-tert-Butoxycarbonylamino-propionylamino)-4-(2,4,5-trifluoro-phenyl)-butyric acid methyl ester (Compound of formula 34) (4.7 gm, 0.0112 mol) in THF, MeOH, water and LiOH H₂O (0.566 gm, 0.0134 mol) were added at room temperature and stirred for 3-hours. After being stirred, THF was evaporated, residue was taken into water, pH of the solution was adjusted to acidic with 1N HCl and aq. layer was extracted with dichloromethane. The combined organic layers were dried over Na₂SO₄ and concentrated under vacuum to give 3-(2-tert-Butoxycarbonylamino-propionylamino)-4-(2,4,5-trifluoro-phenyl)-butyric acid (Compound of formula 35). (Yield=4.5 gm, 96%).
Step-III: Preparation of {1-[3-Oxo-1-(2,4,5-trifluoro-benzyl)-3-(3-trifluoromethyl-5,6-dihvdro-8H-[1,2,4]triazolo[4,3-a]pyrazin-7-yl)propylcarbamoyl]-ethyl}-carbamic acid tert-butyl ester (Compound of formula 36) from 3-(2-tert-Butoxycarbonylamino-propionylamino)-4-(2,4,5-trifluoro-phenyl)-butyric acid (Compound of formula 35):

To a stirred solution of 3-(2-tert-Butoxycarbonylamino-propionylamino)-4-(2,4,5-trifluoro-phenyl)-butyric acid (Compound of formula 35) (4.5 gm, 0.0111 mol) in THF, triazolopyrazine derivative (Compound-7) (2.7 gm, 0.0133 mol), HOBT, triethylamine and N,N′-dicylohexyldicarbodiimide (DCC) were added at 0° C. and stirred for overnight at room temperature. After being stirred, THF was evaporated and residue was taken into water. The aqueous layer was extracted with EtOAc (150 ml). The combined EtOAc layers were washed with 1N HCl and aqueous NaHCO₃. The layers were dried over Na₂SO₄ and concentrated under vacuum to give crude. The crude was purified by coloumn chromatography to give {1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-ethyl}-carbamic acid tert-butyl ester (Compound of formula 36). (Yield=5.3 gm, 82%).
Step-IV: Preparation of 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-propionamide (Compound of formula 37) from {1-[3-Oxo-1-(2,4,5-trifluoro-benzyl)-3-(3-trifluoromethyl-5,6-dihydro-8H-[1,2,4]triazolo[4,3-a]pyrazin-7-yl)propylcarbamoyl]-ethyl}-carbamic acid tert-butyl ester (Compound of formula 36):

{1-[3-Oxo-1-(2,4,5-trifluoro-benzyl)-3-(3-trifloromethyl-5,6-dihydro-8H-[1,2,4]triazolo[4,3-a]pyrazin-7-yl)-propylcarbamoyl]-ethyl}-carbamic acid tert-butyl ester (Compound of formula 36) (5.3 gm), EtOAc-HCl (40 ml) was added at room temperature and stirred for 2-3 hours. After being stirred, The reaction mixture was concentrated and separated 2 gm 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]propionamide hydrochloric acid.

To the 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-propionamide hydrochloric acid, Na₂CO₃ was added and stirred for few minutes. pH of the solution was added above 7 and extracted by MDC followed by dried over Na₂SO₄. The Solution mixture was concentrated and solidified by ether treatment to give crude. The crude was dried under high vacuum to give 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-propionamide (Compound of formula 37). (Yield=4.8 gm, 97.95%)
Step-V: Preparation (1-Benzyl-2-{1-[3-oxo-1-(2,4,5-trifluoro-benzyl)-3-(3-trifluoromethyl-5,6-dihydro-8H-[1,2,4]triazolo[4,3-a]pyrazin-7-yl)propylcarbamoyl]-ethylamino}-allyl)-carbamic acid tert-butyl ester (Compound of formula 38) from 2-Amino-N-[3-oxo-1-(2,4,5-trifluoro-benzyl)-3-(3-trifluoromethyl-5,6-dihydro-8H-[1,2,4]triazolo[4,3-a]pvrazin-7-yl)-propyl]-propionamide (Compound of formula 37):

To a stirred solution of THF and DIPEA, HOBT (0.94 gm), N-Boc-Phe-Alanine (1.4 gm, 0.0052 mol) and DIPEA were added at 0° C. and reaction mixture was stirred after 10 minutes at 0° C. To this stirred reaction mixture, EDC-HCl (1.3 gm) was added at 0° C. and reaction mixture was stirred after 10 minutes at room temperature. 2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-propionamide (Compound of formula 37) (2.5 gm) was added into the reaction mixture and stirred at room temperature for over night. After being stirred, the reaction mixture was concentrated and 1N HCl was added to the concentrated reaction mixture. Then reaction mixture was filtered and washed with aqueous NaHCO₃ and dried. The mixture was washed with hexane (50 ml) and ether (10 ml) and dried under high vacuum to give (1-Benzyl-2-{1-[3-oxo-1-(2,4,5-trifluoro-benzyl)-3-(3-trifluoromethyl-5,6-dihydro-8H-[1,2,4]triazolo [4,3-a]pyrazin-7-yl)-propylcarbamoyl]-ethylamino}-allyl)-carbamic acid tert-butyl ester (Compound of formula 38) as white crude. (Yield=3.13 gm, 82.36%).
STEP-VI: Preparation of 2-Amino-N-{1-[3-oxo-1-(2,4,5-trifluoro-benzyl)-3-(3-trifluoromethyl-5,6-dihydro-8H-[1,2,4]triazolo[4,3-a]pyrazin-7-yl)propylcarbamoyl]-ethyl}-3-phenyl-propionamide (Compound of formula 39) from (1-{1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-ethylcarbamoyl}-2-phenyl-ethyl)-carbamic acid tert-butyl ester (Compound of formula 38):

To (1-{1-[3-Oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-ethylcarbamoyl}-2-phenyl-ethyl)-carbamic acid tert-butyl ester (Compound of formula 38) (3 gm), EtOAc (50 ml) was added at room temperature and stirred for 2 hours. After being stirred, ether (20 ml) was added and stirred for 10-15 minutes to give white residue. The residue was filtered and washed with ether (10 ml) and dried under high vacuum to give 2-Amino-N-{1-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-ethyl}-3-phenyl-propionamide (Compound of formula 39) (Yield=2.67 gm, 97.80%).

In accordance with the present invention the following novel compounds were synthesized according the process described above and these compound were tested for the in vivo screening to evaluate their anti diabetic activity.

| | | |
|---|---|---|
| CPL-2009-0005 | 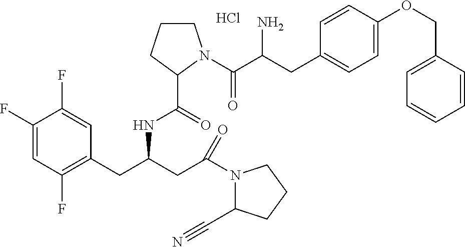 [SMILES: N#CC1CCCN1C(=O)C[C@@H](Cc1cc(F)c(F)cc1F)NC(=O)C1CCCN1C(=O)C(N)Cc1ccc(OCc2ccccc2)cc1 ; Cl] | 1-[2-Amino-3-(4-benzyloxy- phenyl)-propionyl]-pyrrolidine-2- carboxylic acid [3-(2-cyano- pyrrolidin-1-yl)-3-oxo-1-(2,4,5- trifluoro-benzyl)-propyl]-amide hydrochloric acid |
| CPL-2009-0033 | 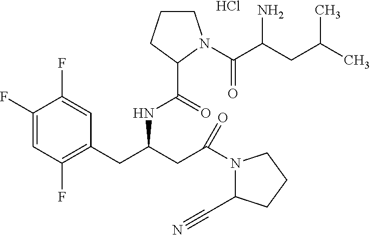 [SMILES: Cl ; CC(C)CC(N)C(=O)N1CCCC1C(=O)N[C@@H](CC(=O)N1CCCC1C#N)Cc1cc(F)c(F)cc1F] | 1-(2-Amino-4-methyl-pentanoyl)- pyrrolidine-2-carboxylic acid [3-(2- cyano-pyrrolidin-1-yl)-3-oxo-1- (2,4,5-trifluoro-benzyl)-propyl]- amide hydrochloric Acid |
| CPL-2009-0029 | 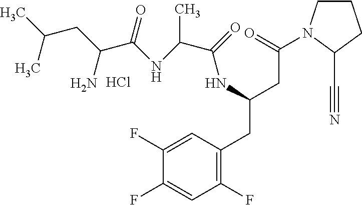 [SMILES: CC(C)CC(N)C(=O)NC(C)C(=O)N[C@@H](CC(=O)N1CCCC1C#N)Cc1cc(F)c(F)cc1F ; Cl] | 2-Amino-4-methyl-pentanoic acid {1-[3-(2-cyano-pyrrolidin-1-yl)-3- oxo-1-(2,4,5-trifluoro-benzyl)- propylcarbamoyl]-ethyl}-amide hydrochloric acid |
| CPL-2009-0027 | 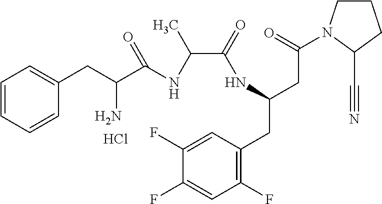 [SMILES: CC(NC(=O)C(N)Cc1ccccc1)C(=O)N[C@@H](CC(=O)N1CCCC1C#N)Cc1cc(F)c(F)cc1F ; Cl] | 2-Amino-N-{1-[3-(2-cyano- pyrrolidin-1-yl)-3-oxo-1-(2,4,5- trifluoro-benzyl)- propylcarbamoyl]-ethyl}-3-phenyl- propionamide hydrochloric Acid |
| CPL-2009-0031 | 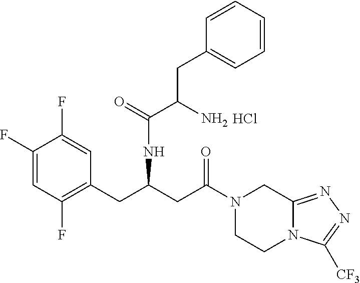 [SMILES: NC(Cc1ccccc1)C(=O)N[C@@H](CC(=O)N1CCn2c(nnc2C(F)(F)F)C1)Cc1cc(F)c(F)cc1F ; Cl] | 2-Amino-N-[3-oxo-1-(2,4,5- trifluoro-benzyl)-3-(3- [1,2,4]triazolo[4,3-a]pyrazin-7-yl)- propyl]-3-phenyl-propionamide hydrochloric acid |
| CPL-2009-0079 | 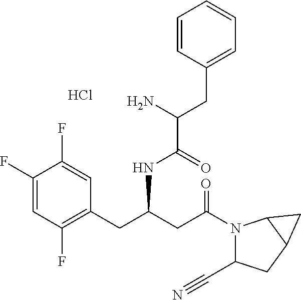 [SMILES: N#CC1CC2CC2N1C(=O)C[C@@H](Cc1cc(F)c(F)cc1F)NC(=O)C(N)Cc1ccccc1 ; Cl] | 2-Amino-N-[3-(3-cyano-2-aza- bicyclo[3.1.0]hex-2-yl)-3-oxo-1- (2,4,5-trifluoro-benzyl)-propyl]-3- phenyl-propionamide hydrochloric Acid |
| CPL-2011-0089 | 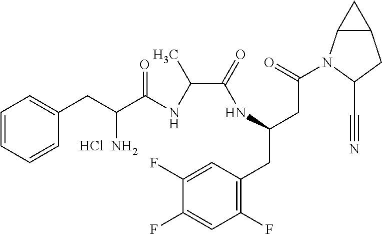 [SMILES: CC(NC(=O)C(N)Cc1ccccc1)C(=O)N[C@@H](CC(=O)N1C(C#N)CC2CC21)Cc1cc(F)c(F)cc1F ; Cl] | 2-Amino-N-{-[3-(3-cyano-2-aza- bicyclo[3.1.0]hex-2-yl)-3-oxo-1- (2,4,5-trifluoro-benzyl)- propylcarbamoyl]-ethyl]-3-phenyl- propionamide hydrochloric Acid |
| CPL-2011-0103 | 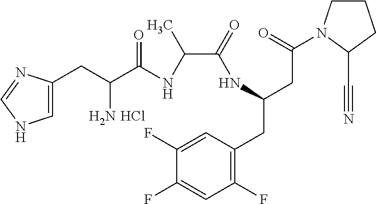 [SMILES: CC(NC(=O)C(N)Cc1c[nH]cn1)C(=O)N[C@@H](CC(=O)N1CCCC1C#N)Cc1cc(F)c(F)cc1F ; Cl] | 2-Amino-N-{-[3-(2-cyano- pyrrolidin-1-yl)-3-oxo-1-(2,4,5- trifluoro-benzyl)- propylcarbamoyl]-ethyl]-3-(1H- imidazol-4-yl)-propionamide hydrochloric Acid |

### General Procedure For in Vivo Screening

The novel hypoglycemic compounds according to present invention on reaction with DPP IV enzyme release an entity that inhibits the DPP IV enzyme. The inhibition of DPP IV enzyme by compound of formula XII is attributed to the particular amino acid analogues synthesized as per present invention. These are evaluated by the in vivo screening of the compounds of present invention.

### In Vivo Study of Synthesized Compounds:

The efficacy of compounds as per table 1 was evaluated through Oral Glucose Tolerance Test in animals. Each group consist of 10-12 weeks old Wistar female rats. The animals were divided into 4 different groups of 6 animals per group. The group I received vehical (WFI Placebo), Group II, Group III and Group IV compound as per Table 1. All animals had over night fasting. All compounds as per table 1 were administered in equimolar Dose (In mg/kg) of active at 0 hour to all the study group animals with 10 mL/kg of dose volume.

TABLE 01
Dose groups, no. of animals and dose of drugs for in vivo study:

| | No. of | | Dose | Dose volume |
|---|---|---|---|---|
| Group | animals | Drugs | (In mg/kg) | (mL/kg) |
| I | 6 | Normal control | 0.0 | 10 |
| II | 6 | CPL-2009-0027 | 11.7 | 10 |
| III | 6 | CPL-2009-0030 | 11.28 | 10 |
| IV | 6 | CPL-2009-0031 | 12.0 | 10 |

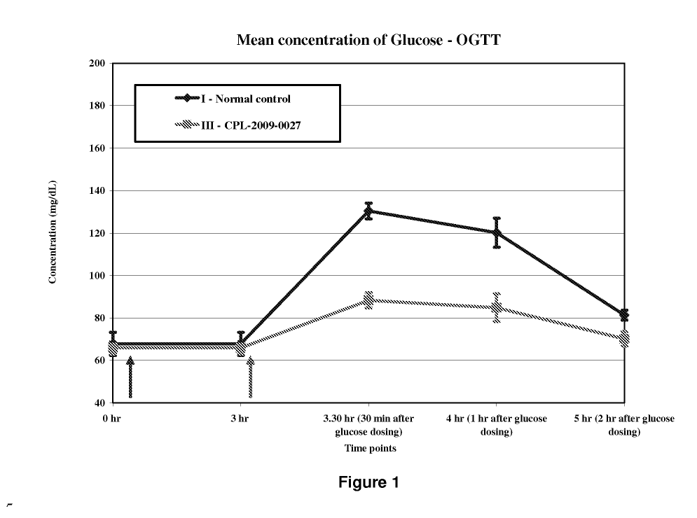

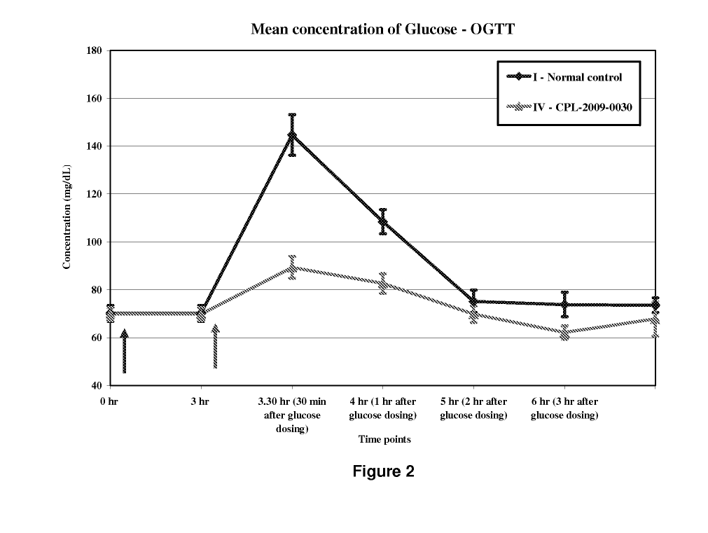

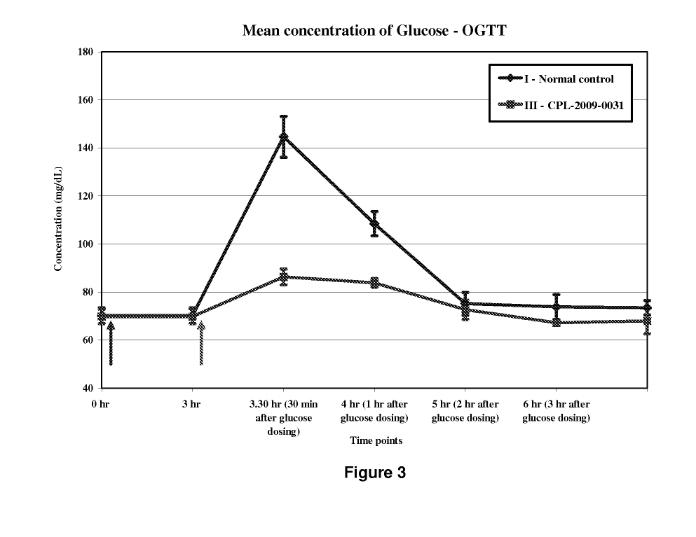

The compounds were administered orally. The glucose (2 gm/Kg body weight) was administered 3 hrs after the administration of compounds. Blood glucose was measured at the time of administration of compound, glucose and 30 min, 1 hr, 2 hr, 3 hr and 4 hr after the glucose administration. The change in glucose levels over time is shown in FIG. 1. All animals showed rise in blood glucose level following administration of glucose. The rise was maximum at 30 min after glucose administration. The rise in glucose is significantly lower when compound of present invention were administered compared to no treatment group. The difference between no treatment group and other three groups is maximum at 30 min and it decreases over time (FIG. 01-03). The findings also show that the compound of present invention does not alter the fasting glucose levels from 0 to 3 Hrs. Thus, the compound of present invention reduces hyperglycaemic effect of glucose without inducing hypoglycaemia. In other words, compounds of present invention provide hypoglycaemic effect only to reduce post-prandial hyperglycaemia without inducing fasting hypoglycaemia.

To confirm the finding the experiment was repeated three times and the percentage change in glucose level over time of three experiments is presented in table 02. The repeat experiments also confirm the findings. The results of these experiments are depicted in FIG. 1-3.

This is further evaluated by following analytical method.

### Analytical Method to Analyze the DPP IV Compounds of Present Invention:

Reagents and Solvents: The reagents, solvents, standards and equipments to analyze the DPP IV compounds of present invention are as under:
   Trifluoro Acetic acid (AR grade)
   Acetonitrile (HPLC grade)
   Milli Q water
   Sitagliptin Base as working standard DPP IV inhibitor
   Shimadzu LC-2010 equipped with UV detector and Auto Sampler
   Diluent: Acetonitrile

### Preparation of Buffer

Transfer accurately measured 1000 mL Milli Q water in a beaker. Adjust pH 2.00±0.05 with Trifluoro acetic acid. Shake it gently and filter through 0.45 μ membrane filter.

Preparation of Mobile phase

Transfer 300 mL acetonitrile in 1000 mL volumetric flask and make the volume up to the mark with buffer pH 2.00±0.05

### Standard Preparation

Exactly weigh about 20 mg control drug as working standard in 10 mL volumetric flask. Add 5.0 mL diluent and sonicate it (if required) to dissolve the solids and make the volume up to the mark with diluent giving a standard solution having concentration 2000 ppm (Stock solution).

Above stock solution was further diluted to get different concentration solution varying from 0.025 μM to 100 μM. Linearity curve of peak area against concentration in μM was plotted for different concentration.

### Sample Preparation

Extracted samples (after removing proteneous matter) were provided from different organs (Liver, Kidney and Pancreas) and serum samples which were directly injected on to HPLC system.

### Chromatographic Conditions:

The liquid chromatography is equipped with variable wavelength UV detector, Auto sampler and Data processor
Column: ypersil BDS C8, 4.6mm×250 mm, 5 μ
Detector wavelength: 54 nm
Flow Rate: 1.0 mL/min
Injection volume: 20 μL
Column Temperature: 60° C.

### Procedure

Inject blank (diluent) and blank extraction samples, inject standard preparation of varying concentration from 0.025 μM to 100 μM and plot a graph of Area under curve against concentration in μM. Inject sample preparation and record the chromatogram. Disregard any peak due to blank and calculate concentration of an entity which is released from the extracted samples collected at different time intervals.

Similarly in identical experiments were performed to evaluate the in vivo screening of compounds as per present invention. The result of in vivo screening for evaluation of antidiabetic effect of CPL-2009-0005, CPL-2009-0033, CPL-2009-0029, CPL-2009-0079, CPL-2011-0089, CPL-2011-0103 and like are also showing inhibition of DPP IV enzyme.

The above results indicate that the novel compounds according to present invention are showing antidiabetic activity when compared with standard drugs.

1. A compound of formula VI, 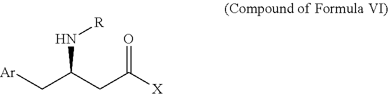
or a pharmaceutically acceptable salt thereof,
wherein, Ar is phenyl or substituted phenyl having 1 to 5 halogen atoms in the phenyl ring;
R is aa1 or aa₁-aa₂ or aa₁-aa₂-aa₃ or aa-aa₂-aa₃-aa₄, wherein aa₁, aa₂, aa₃ and aa₄ are amino acids selected independently and is linked through a peptide bond; wherein each of aa₁, aa₂, aa₃, and aa₄ is independently selected from any of the following: 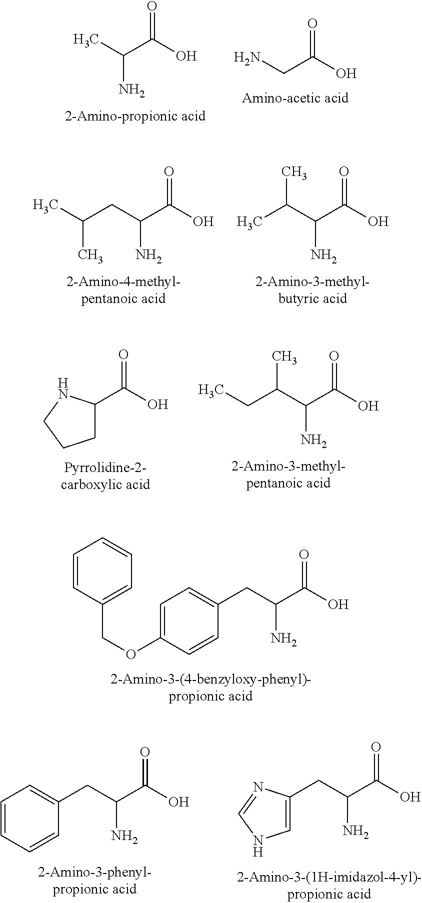
X is defined as any of the substructure X₁ to X₅, wherein substructures X₁ to X₅ are structurally depicted below:

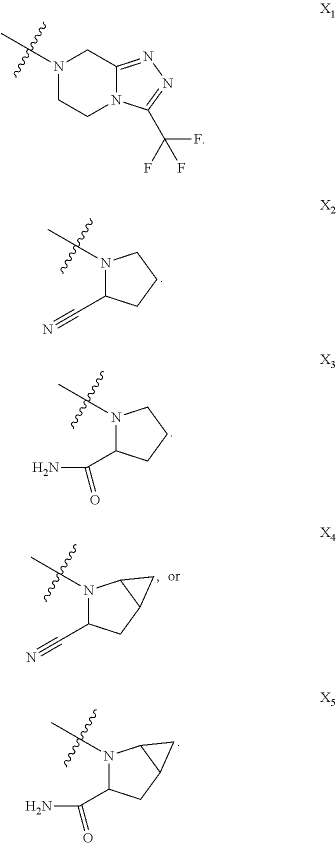 [SMILES: *N1CCn2c(nnc2C(F)(F)F)C1 ; *N1CCCC1C#N ; *N1CCCC1C(N)=O ; *N1C(C#N)CC2CC21 ; *N1C(C(N)=O)CC2CC21]

2. The compound as claimed in claim 1 having formula XII,
(Compound of Formula XII) 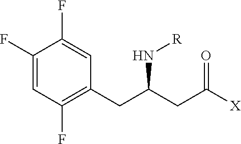
wherein,

| | | —X is any one from X₁₋₅ | | | | |
|---|---|---|---|---|---|---|
| Nₐ₋ₑ | —R | Nₐ = X₁ | N_{b} = X₂ | N_{c} = X₃ | N_{d} = X₄ | Nₑ = X₅ |
| 1ₐ₋ₑ | 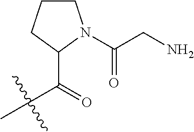 [SMILES: *C(=O)C1CCCN1C(=O)CN] | X₁ | X₂ | X₃ | X₄ | X₅ |
| 2ₐ₋ₑ | 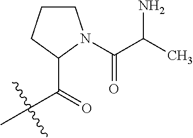 [SMILES: *C(=O)C1CCCN1C(=O)C(C)N] | | X₂ | X₃ | X₄ | X₅ |
| 3ₐ₋ₑ | 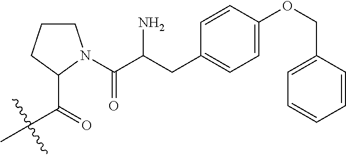 [SMILES: *C(=O)C1CCCN1C(=O)C(N)Cc1ccc(OCc2ccccc2)cc1] | X₁ | X₂ | X₃ | X₄ | X₅ |
| 4ₐ₋ₑ | 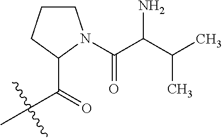 [SMILES: *C(=O)C1CCCN1C(=O)C(N)C(C)C] | | X₂ | X₃ | X₄ | X₅ |
| 5ₐ₋ₑ | 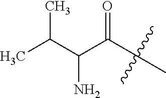 [SMILES: *C(=O)C(N)C(C)C] | | X₂ | X₃ | X₄ | X₅ |
| 6ₐ₋ₑ | 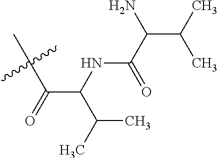 [SMILES: *C(=O)C(NC(=O)C(N)C(C)C)C(C)C] | X₁ | X₂ | X₃ | X₄ | X₅ |
| 7ₐ₋ₑ | 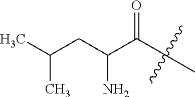 [SMILES: *C(=O)C(N)CC(C)C] | | X₂ | X₃ | X₄ | X₅ |
| 8ₐ₋ₑ | 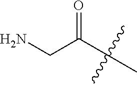 [SMILES: *C(=O)CN] | X₁ | X₂ | X₃ | X₄ | X₅ |
| 9ₐ₋ₑ | 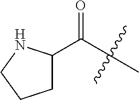 [SMILES: *C(=O)C1CCCN1] | | X₂ | X₃ | X₄ | X₅ |
| 10ₐ₋ₑ | 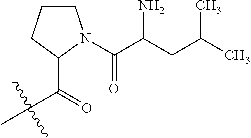 [SMILES: *C(=O)C1CCCN1C(=O)C(N)CC(C)C] | | X₂ | X₃ | X₄ | X₅ |
| 11ₐ₋ₑ | 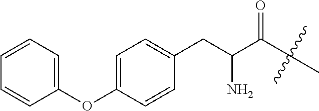 [SMILES: *C(=O)C(N)Cc1ccc(Oc2ccccc2)cc1] | | X₂ | X₃ | X₄ | X₅ |
| 12ₐ₋ₑ | 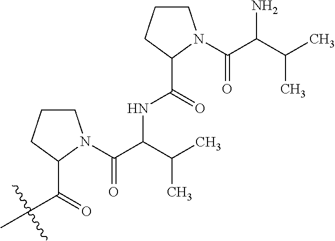 [SMILES: *C(=O)C1CCCN1C(=O)C(NC(=O)C1CCCN1C(=O)C(N)C(C)C)C(C)C] | | X₂ | X₃ | X₄ | X₅ |
| 13ₐ₋ₑ | 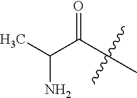 [SMILES: *C(=O)C(C)N] | | X₂ | X₃ | X₄ | X₅ |
| 14ₐ₋ₑ | 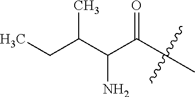 [SMILES: *C(=O)C(N)C(C)CC] | X₁ | X₂ | X₃ | X₄ | X₅ |
| 15ₐ₋ₑ | 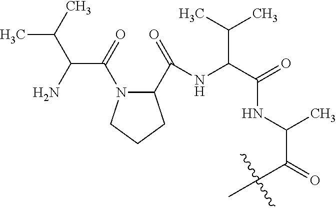 [SMILES: *C(=O)C(C)NC(=O)C(NC(=O)C1CCCN1C(=O)C(N)C(C)C)C(C)C] | X₁ | X₂ | X₃ | X₄ | X₅ |
| 16ₐ₋ₑ | 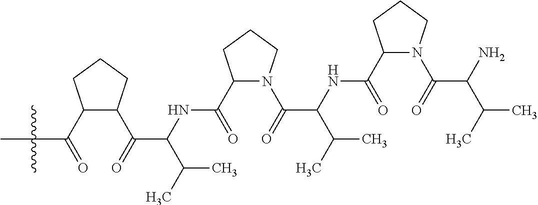 [SMILES: *C(=O)C1CCCC1C(=O)C(NC(=O)C1CCCN1C(=O)C(NC(=O)C1CCCN1C(=O)C(N)C(C)C)C(C)C)C(C)C] | X₁ | X₂ | X₃ | X₄ | X₅ |
| 17ₐ₋ₑ | 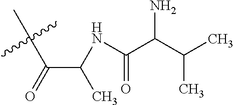 [SMILES: *C(=O)C(C)NC(=O)C(N)C(C)C] | | X₂ | X₃ | X₄ | X₅ |
| 18ₐ₋ₑ | 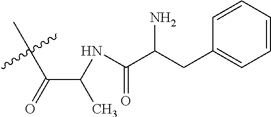 [SMILES: *C(=O)C(C)NC(=O)C(N)Cc1ccccc1] | X₁ | X₂ | X₃ | X₄ | X₅ |
| 19ₐ₋ₑ | 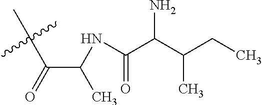 [SMILES: *C(=O)C(C)NC(=O)C(N)C(C)CC] | X₁ | X₂ | X₃ | X₄ | X₅ |
| 20ₐ₋ₑ | 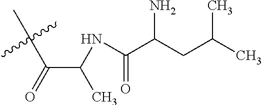 [SMILES: *C(=O)C(C)NC(=O)C(N)CC(C)C] | X₁ | X₂ | X₃ | X₄ | X₅ |
| 21ₐ₋ₑ | 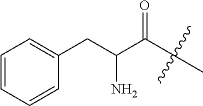 [SMILES: *C(=O)C(N)Cc1ccccc1] | | | X₃ | X₄ | X₅ |
| 22ₐ₋ₑ | 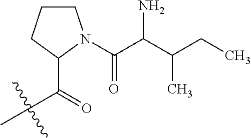 [SMILES: *C(=O)C1CCCN1C(=O)C(N)C(C)CC] | X₁ | X₂ | X₃ | X₄ | X₅ |
| 23ₐ₋ₑ | 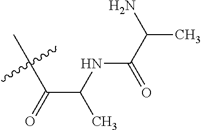 [SMILES: *C(=O)C(C)NC(=O)C(C)N] | X₁ | X₂ | X₃ | X₄ | X₅ |
| 24ₐ₋ₑ | 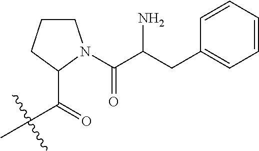 [SMILES: *C(=O)C1CCCN1C(=O)C(N)Cc1ccccc1] | X₁ | X₂ | X₃ | X₄ | X₅ |
| 25ₐ₋ₑ | 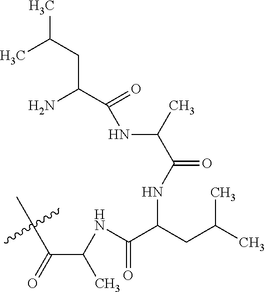 [SMILES: *C(=O)C(C)NC(=O)C(CC(C)C)NC(=O)C(C)NC(=O)C(N)CC(C)C] | X₁ | X₂ | X₃ | X₄ | X₅ |
| 26ₐ₋ₑ | 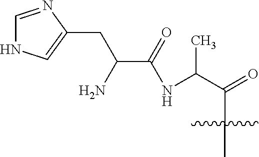 [SMILES: *C(=O)C(C)NC(=O)C(N)Cc1c[nH]cn1] | X₁ | X₂ | X₃ | X₄ | X₅ |

and X₁₋₅ is as defined in claim 1;
or a pharmaceutically acceptable salt thereof.

3. A process for the preparation of DPP-IV inhibitors of formula VI, 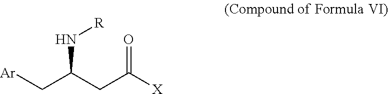
using sequence of reactions as depicted in scheme 1 comprises:
a. deprotection followed by esterification of N-protected beta-amino acid (formula I) to give chiral beta amino acid derivatives (formula II);
b. reaction of chiral beta amino acid derivatives (formula II) with carboxyl terminal N′-protected peptide compounds to give beta-amino N-acetyl peptide compounds (formula III);
c. reaction of beta-amino N-protected compound (formula III) with saponifying agent to provide beta-amino N-acylated-4-(2,4,5-trifluorophenyl)-3-amino butyric acid (formula IV);
d. condensation of beta-amino N-acylated-4-(2,4,5-trifluorophenyl)-3-amino butyric acid (formula IV) with any of the compound (a) to (e) to give corresponding amides of beta-amino N-acylated-N′-protected peptide compounds (formula V);
e. deprotection of amides of beta-amino N-acylated-N′-protected peptide compounds (formula V) to give 4-aryl-3-N-peptide substituted amino acid derivatives (formula VI).

4. A process for the preparation of novel DPP-IV inhibitor of formula XII, using sequence of reactions as depicted in scheme 2 comprises: 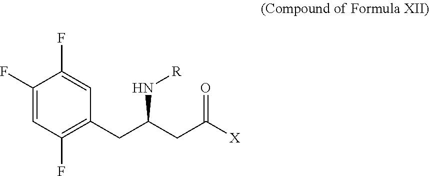
a. deprotection followed by esterification of N-protected beta-amino acid (formula VII) to give chiral beta amino acid derivatives (formula VIII);
b. reaction of chiral beta amino acid derivative (formula VIII) with carboxyl terminal N′-protected peptide compounds to give beta-amino N-acylated peptide compounds (formula IX), wherein As is 2,4,5-trifluoro phenyl;
c. beta-amino N-acylated peptide compounds (formula IX) are converted into beta-amino N-acylated-4-(2,4,5-trifluorophenyl) butanoic acids (formula X) by saponification;
d. reacting compounds of formula (X) with any of the compound (a) to (e) to give corresponding amides of formula (XI);
e. deprotection of beta-amino N-acylated-N′-protected peptide compounds of formula (XI) to give 4-aryl-3-N-peptide substituted amino acid derivatives (formula XII).

5. The process as claimed in claim 3, wherein Peptide bond is formed by coupling between a carboxyl group and an amino group in presence of coupling agents.

6. The coupling agents as claimed in claim 5 are selected from DCC, EDC, DIC and like.

7. The process as claimed in claim 3, the saponification reaction is carried out by using Inorganic base.

8. The process as claimed in claim 7, wherein the inorganic base is selected from NaOH, KOH, LiOH and like, preferably NaOH and LiOH.

9. The compound of claim 2 having a formula XII a 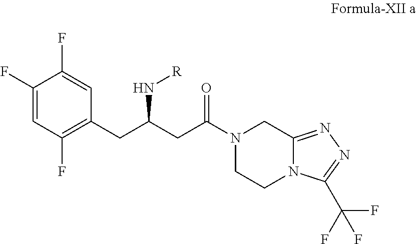
wherein R is selected from R₁₋₂₆: 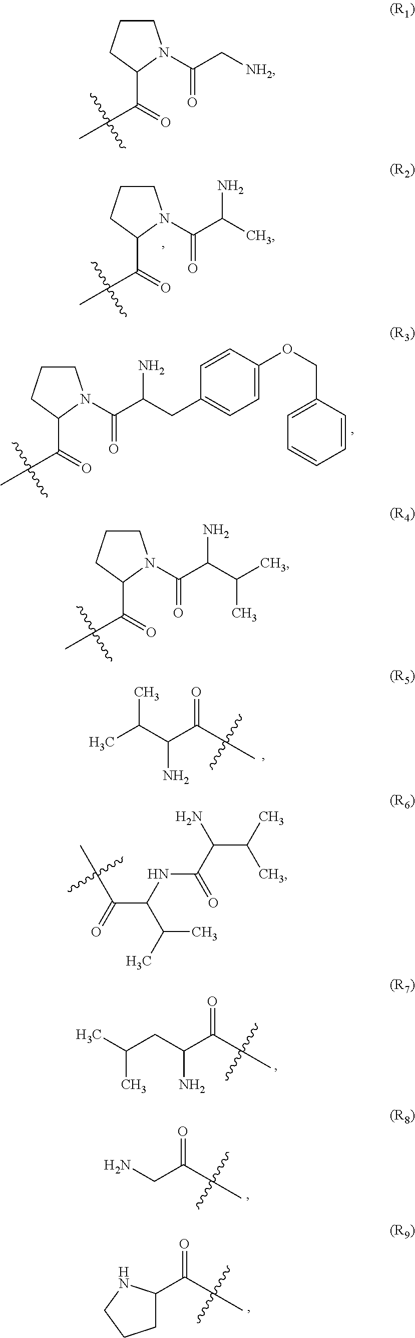 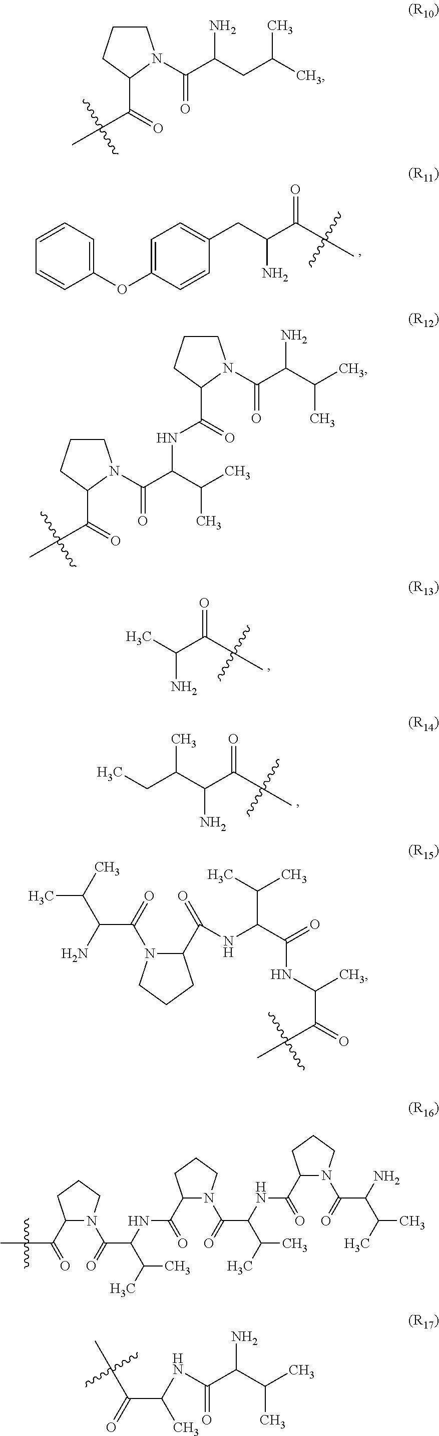 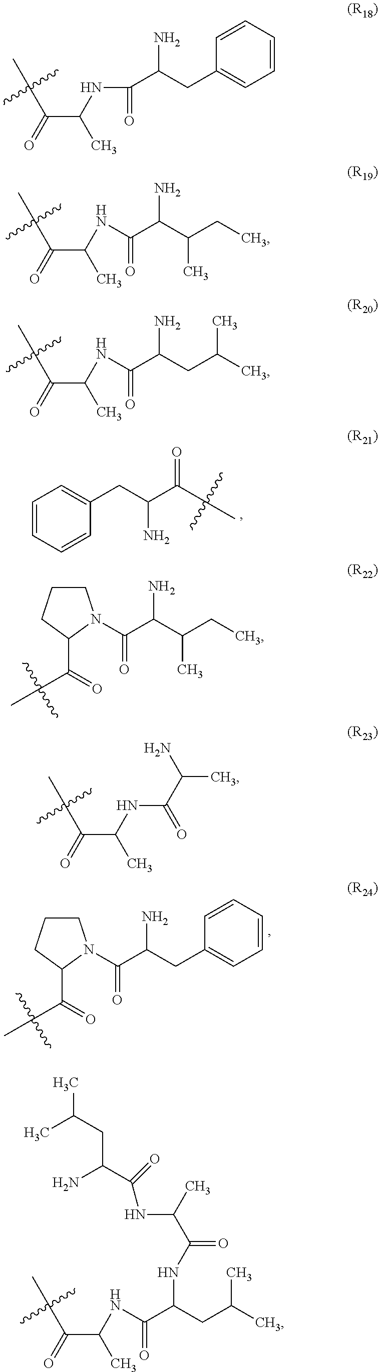 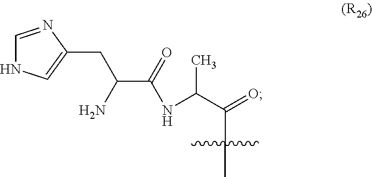 [SMILES: *C(=O)C(C)NC(=O)C(N)Cc1c[nH]cn1]
or a pharmaceutically acceptable salt thereof.

10. The compound of claim 2 having a formula XII b 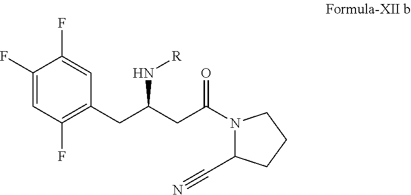
wherein R is selected from R₁₋₂₆: 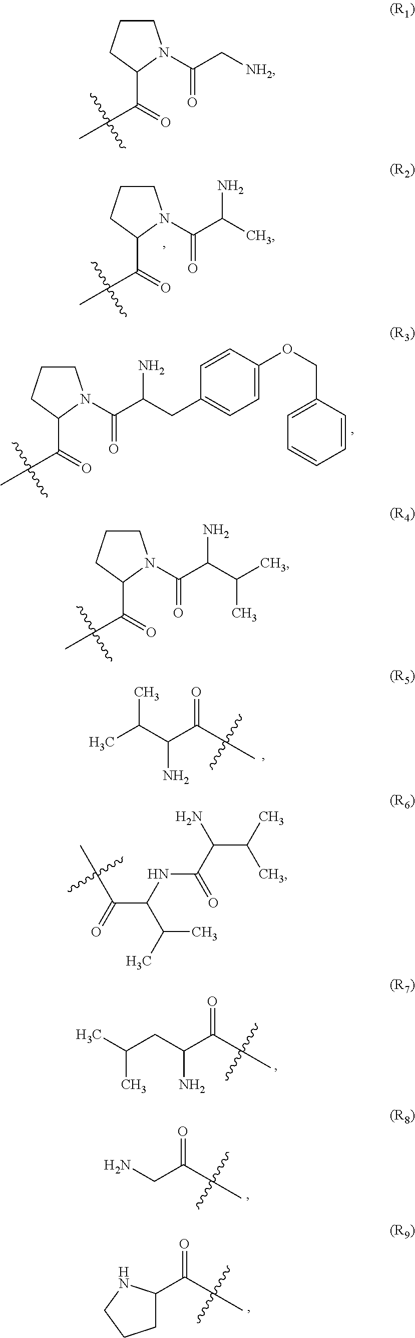 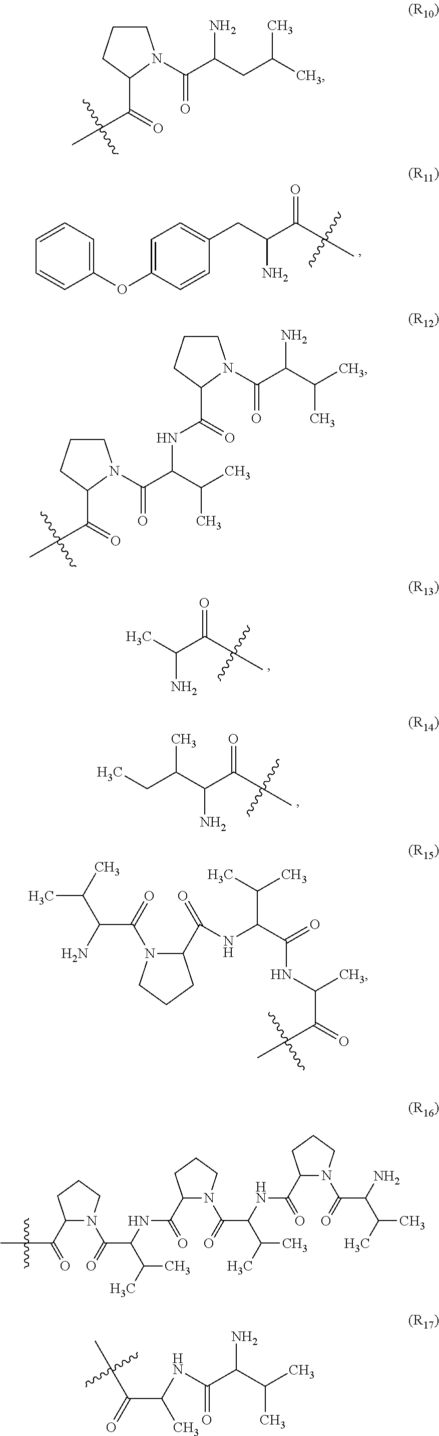 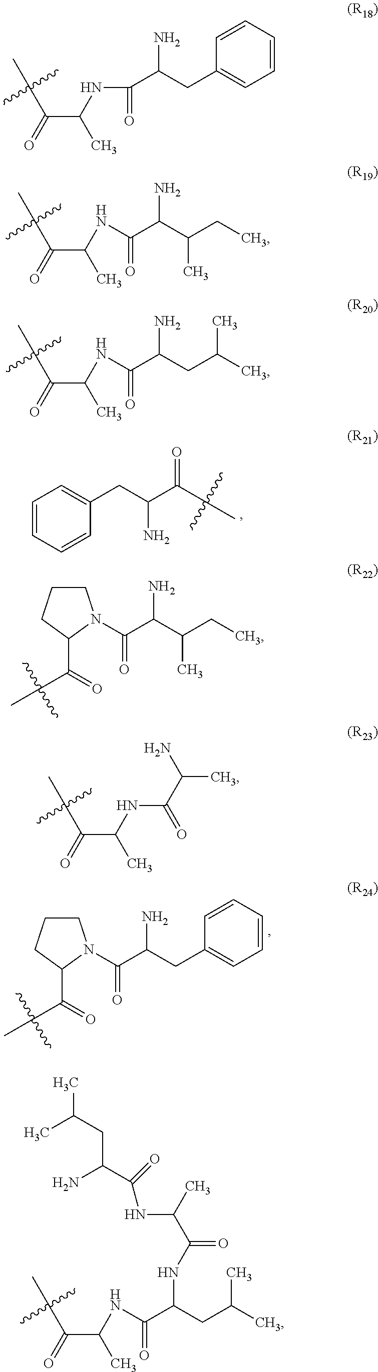 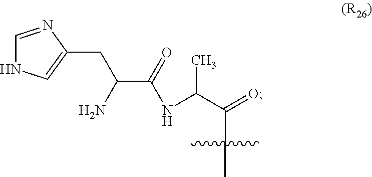 [SMILES: *C(=O)C(C)NC(=O)C(N)Cc1c[nH]cn1]
or a pharmaceutically acceptable salt thereof.

11. The compound of claim 2 having a formula XII c 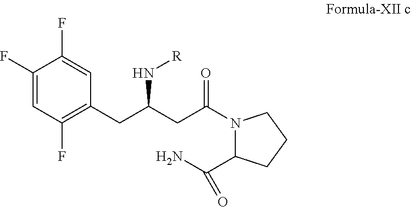
wherein R is selected from R₁₋₂₆: 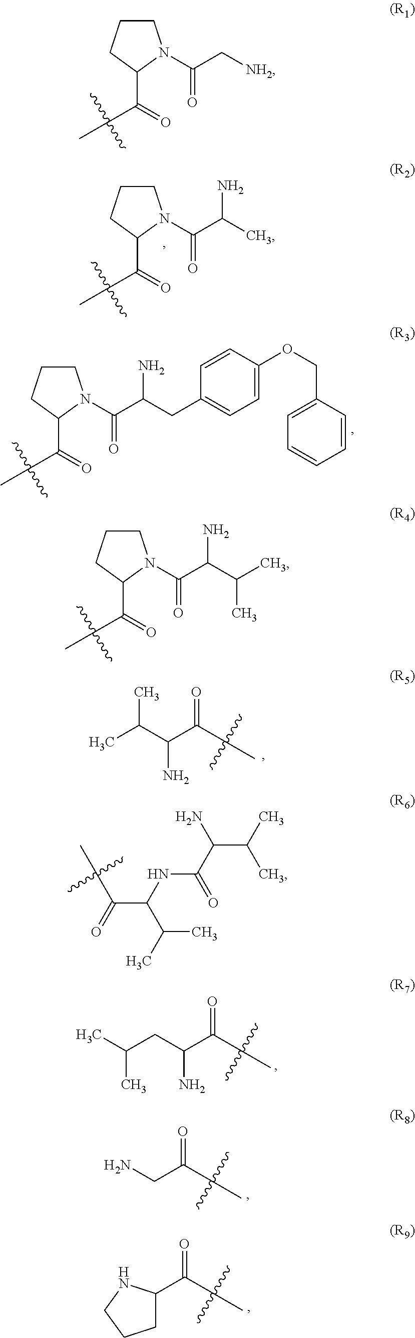 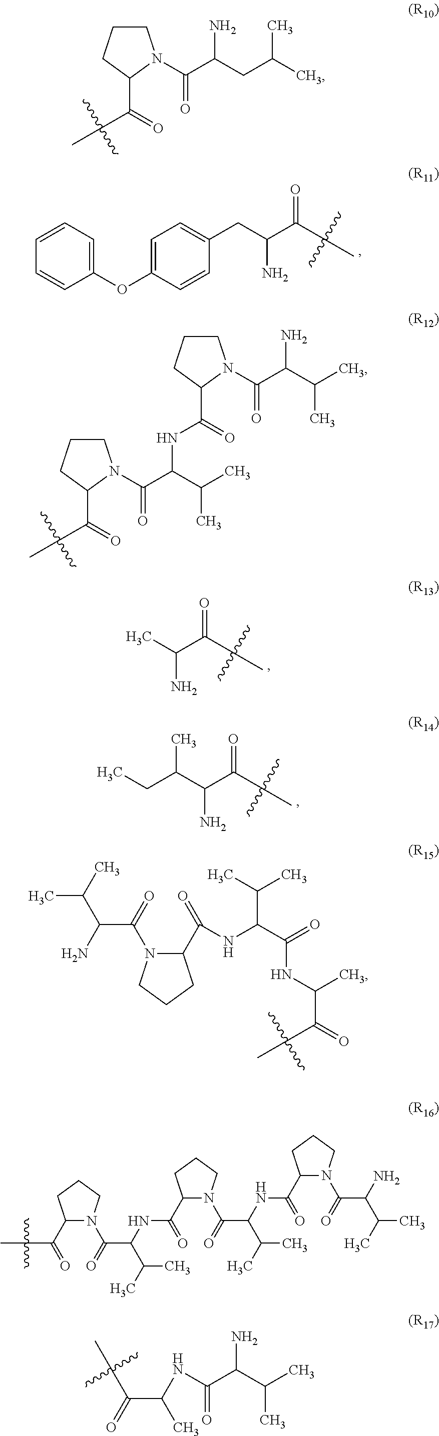 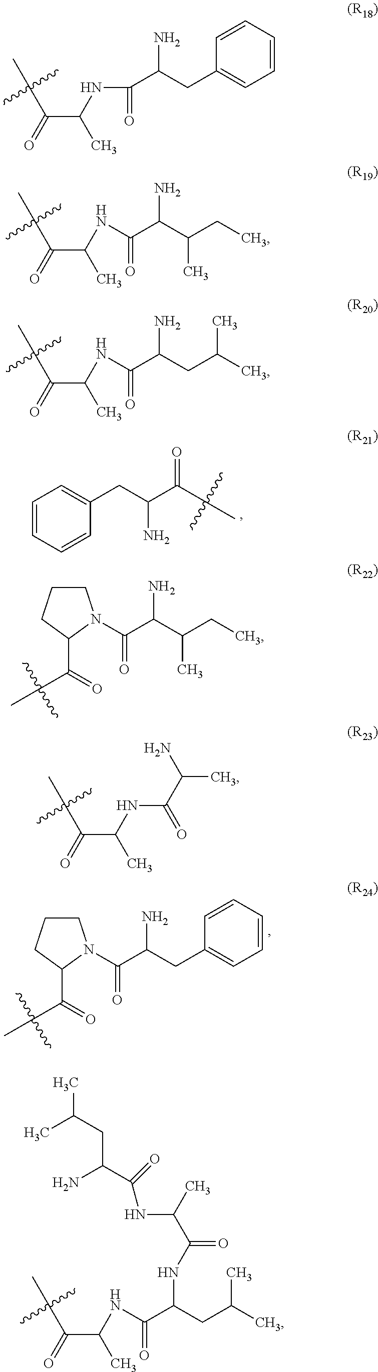 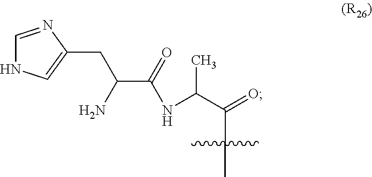 [SMILES: *C(=O)C(C)NC(=O)C(N)Cc1c[nH]cn1]
or a pharmaceutically acceptable salt thereof.

12. The compound of claim 2 having a formula XII d 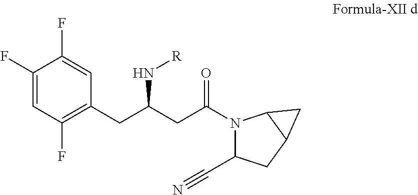
wherein R is selected from R₁₋₂₆: 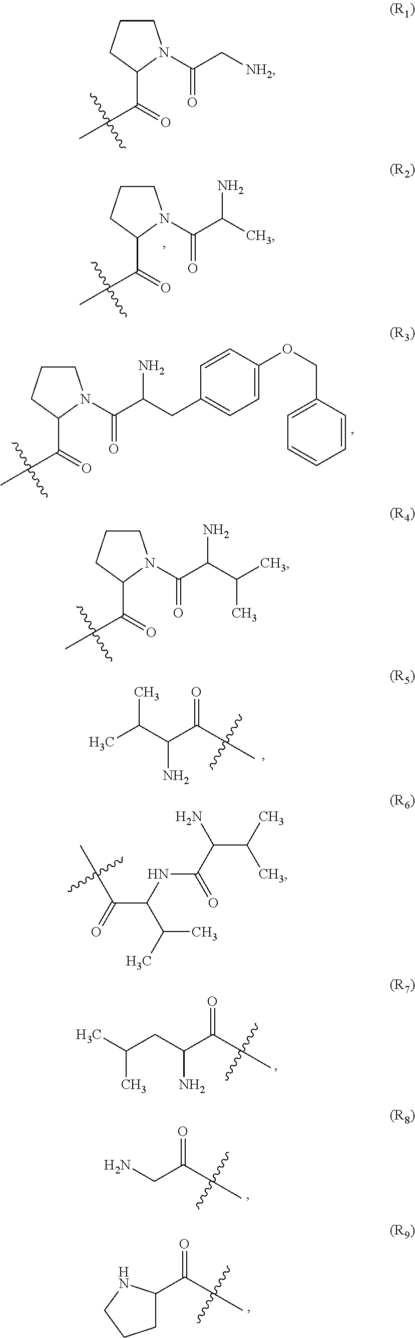 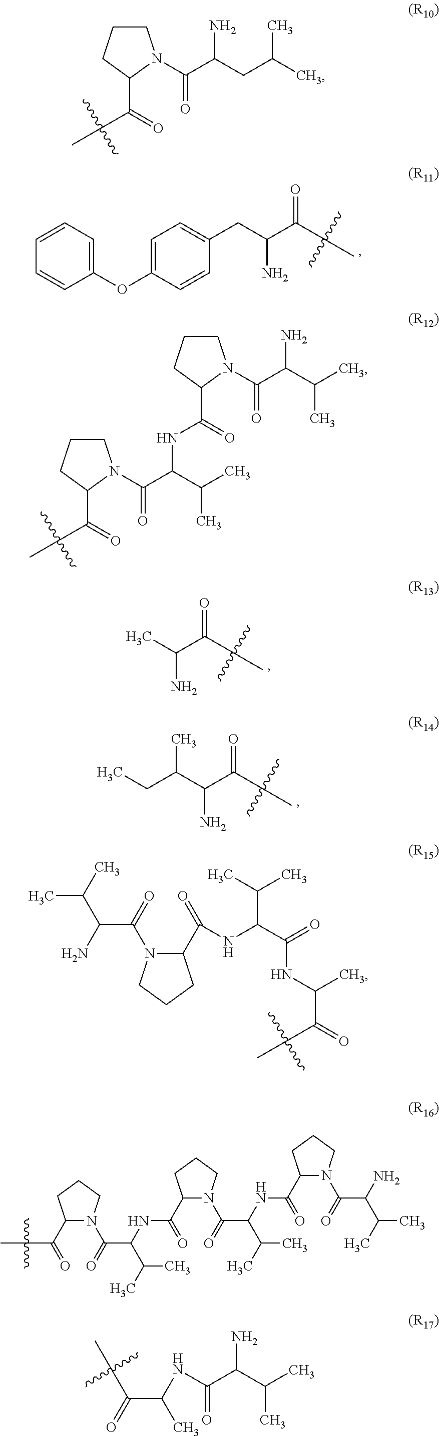 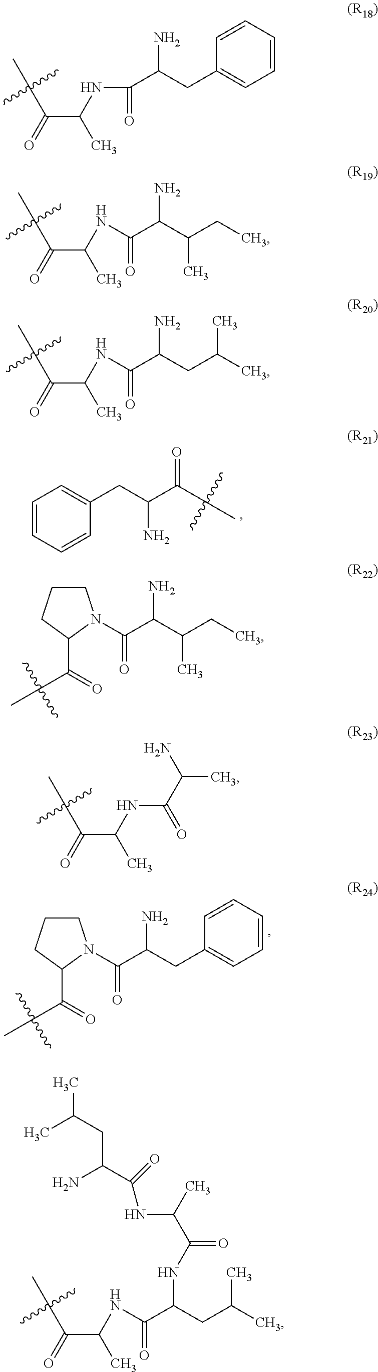 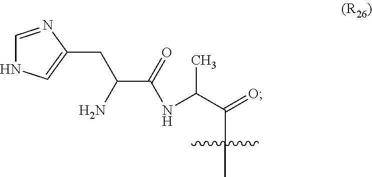 [SMILES: *C(=O)C(C)NC(=O)C(N)Cc1c[nH]cn1]
or a pharmaceutically acceptable salt thereof.

13. The compound of claim 2 having a formula XII e 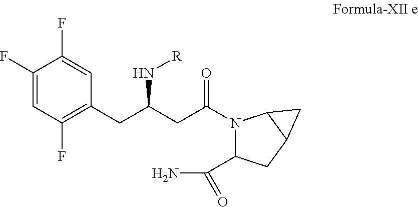
wherein R is selected from R₁₋₂₆: 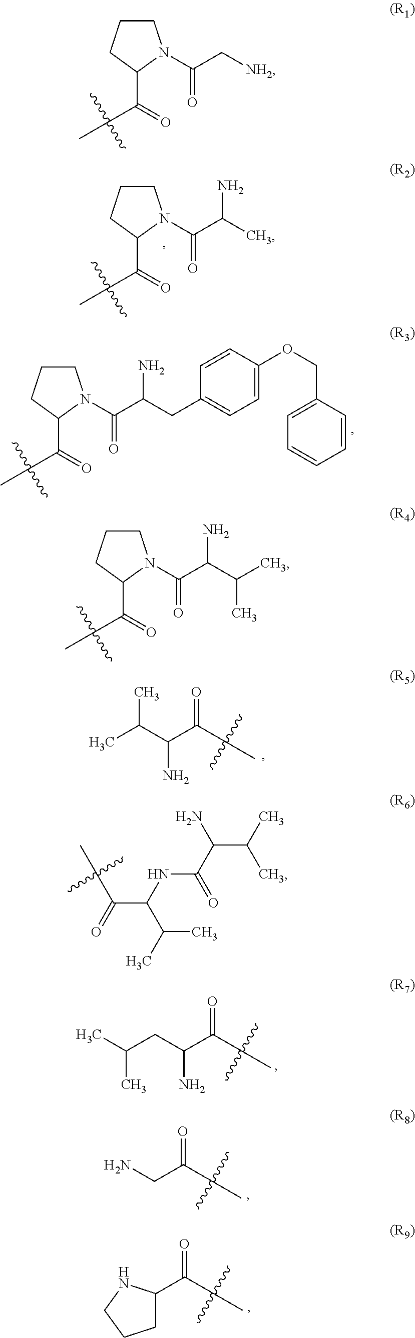 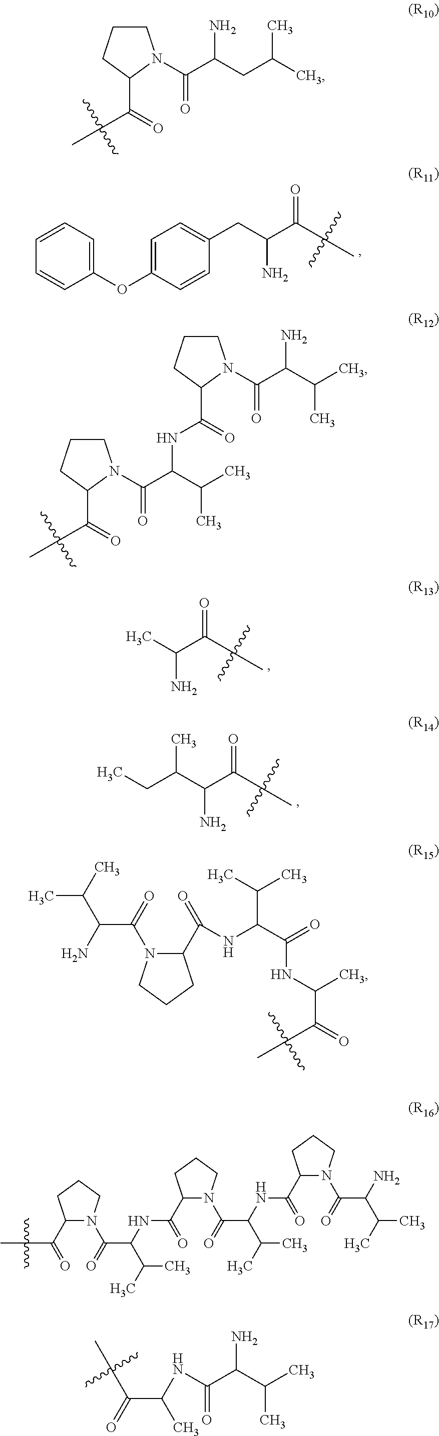 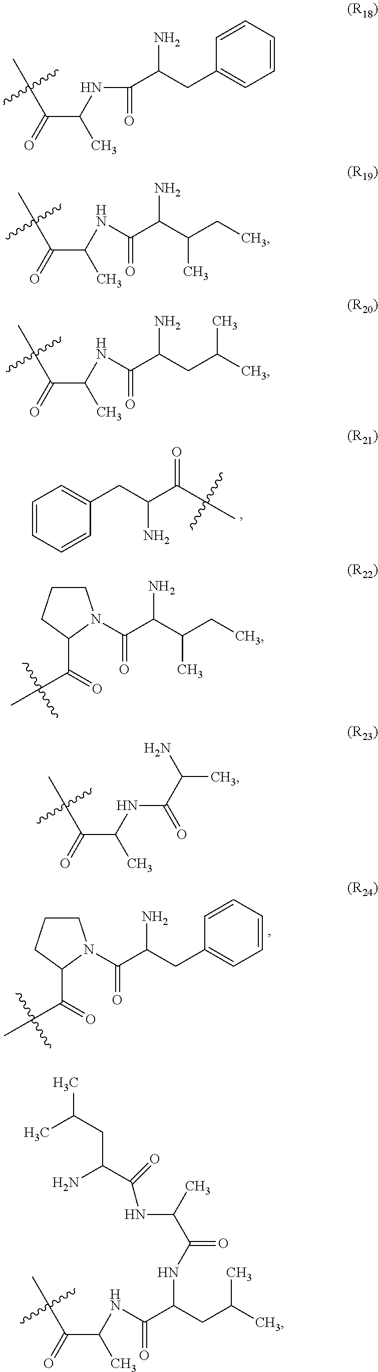 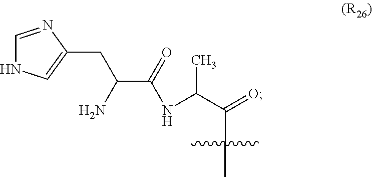 [SMILES: *C(=O)C(C)NC(=O)C(N)Cc1c[nH]cn1]
or a pharmaceutically acceptable salt thereof.

14. The compound according to claim 3, wherein the said compound is selected from the group consisting of:
2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-3-phenyl-propionamide hydrochloric acid: 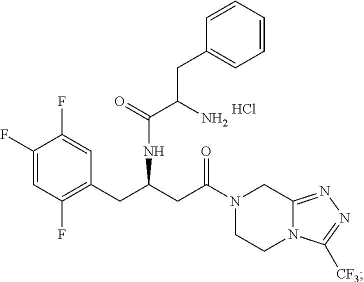 [SMILES: NC(Cc1ccccc1)C(=O)N[C@@H](CC(=O)N1CCn2c(nnc2C(F)(F)F)C1)Cc1cc(F)c(F)cc1F ; Cl]
2-Amino-3-methyl-pentanoic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide hydrochloric acid 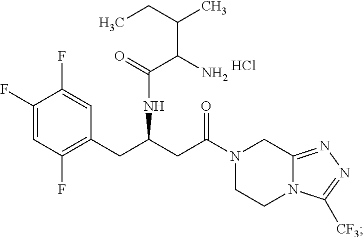 [SMILES: CCC(C)C(N)C(=O)N[C@@H](CC(=O)N1CCn2c(nnc2C(F)(F)F)C1)Cc1cc(F)c(F)cc1F ; Cl]
1-(2-Amino-3-methyl-butyryl)-pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]-amide hydrochloric acid 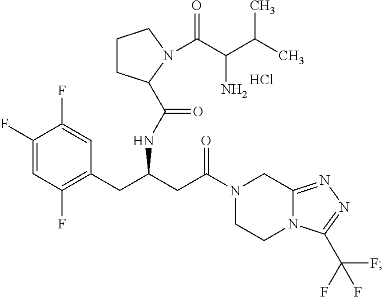 [SMILES: CC(C)C(N)C(=O)N1CCCC1C(=O)N[C@@H](CC(=O)N1CCn2c(nnc2C(F)(F)F)C1)Cc1cc(F)c(F)cc1F ; Cl]
1-(2-Amino-3-phenyl-propionyl)-pyrrolidine-2-carboxylic acid [3-oxo-1-(2,4,5-trifluoro-benzyl)-3-(3-trifluoromethyl-5,6-dihydro-8H-[1,2,4]triazolo[4,3-a]pyrazin-7-yl)-propyl]amide hydrochloric acid 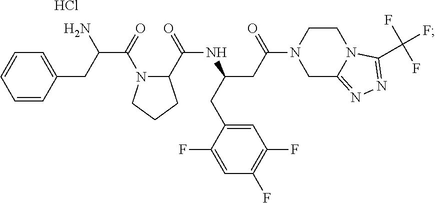 [SMILES: Cl ; NC(Cc1ccccc1)C(=O)N1CCCC1C(=O)N[C@@H](CC(=O)N1CCn2c(nnc2C(F)(F)F)C1)Cc1cc(F)c(F)cc1F]
2-Amino-N-{1-[3-oxo-1-(2,4,5-trifluoro-benzyl)-3-(3-trifluoromethyl-5,6-dihydro-8H-[1,2,4]triazolo[4,3-a]pyrazin-7-yl)-propylcarbamoyl]-ethyl}-3-phenyl-propionamide hydrochloric acid 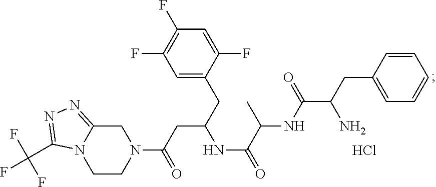 [SMILES: CC(NC(=O)C(N)Cc1ccccc1)C(=O)NC(CC(=O)N1CCn2c(nnc2C(F)(F)F)C1)Cc1cc(F)c(F)cc1F ; Cl]
2-Amino-N-[3-oxo-1-(2,4,5-trifluoro-benzyl)-3-(3-trifluoromethyl-5,6-dihydro-8H-[1,2,4]triazolo[4,3-a]pyrazin-7-yl)-propyl]-3-phenyl-propionamide Phosphoric Acid 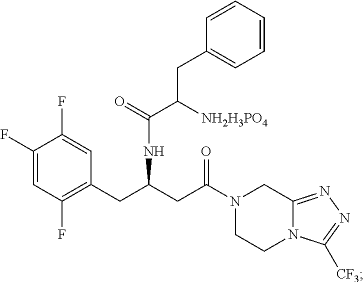
or a pharmaceutically acceptable salt thereof.

15-19. (canceled)

20. The compound as claimed in claims 11 selected from 1-[3-(2-Amino-3-phenyl-propionylamino)-4-(2,4,5-trifluoro-phenyl)-butyryl]-pyrrolidine-2-carboxylic acid amide trifluoro-acetic acid structurally depicted as;

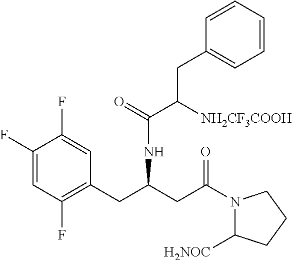

21. The process as claimed in claim 4, wherein Peptide bond is formed by coupling between a carboxyl group and an amino group in presence of coupling agents.

22. The coupling agents as claimed in claim 21 are selected from DCC, EDC, DIC and like.

23. The process as claimed in claim 4, the saponification reaction is carried out by using Inorganic base.

24. The process as claimed in claim 23, wherein the inorganic base is selected from NaOH, KOH, LiOH and like, preferably NaOH and LiOH.
